SEQUENCE LISTING (2 sequences)

SEQ ID NO: 1 - protein, 488 aa, Homo sapiens
MAYSQGGGKKKVCYYYDGDIGNYYYGQGHPMKPHRIRMTHNLLLNYGLYRKMEIYRPHKATAEEMTKYHSDEYIKFLRSIRPDNMSEYSKQMQRFNVGEDCPVFDGLFEFCQLSTGGSVAGAVKLNRQQTDMAVNWAGGLHHAKKSEASGFCYVNDIVLAILELLKYHQRVLYIDIDIHHGDGVEEAFYTTDRVMTVSFHKYGEYFPGTGDLRDIGAGKGKYYAVNFPMRDGIDDESYGQIFKPIISKVMEMYQPSAVVLQCGADSLSGDRLGCFNLTVKGHAKCVEVVKTFNLPLLMLGGGGYTIRNVARCWTYETAVALDCEIPNELPYNDYFEYFGPDFKLHISPSNMTNQNTPEYMEKIKQRLFENLRMLPHAPGVQMQAIPEDAVHEDSGDEDGEDPDKRISIRASDKRIACDEEFSDSEDEGEGGRRNVADHKKGAKKARIEEDKKETEDKKTDVKEEDKSKDNSGEKTDTKGTKSEQLSNP

SEQ ID NO: 2 - protein, 589 aa, Homo sapiens
MDLIDILWRQDIDLGVSREVFDFSQRRKEYELEKQKKLEKERQEQLQKEQEKAFFAQLQLDEETGEFLPIQPAQHIQSETSGSANYSQVAHIPKSDALYFDDCMQLLAQTFPFVDDNEVSSATFQSLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKN

(19) United States
(12) Patent Application Publication
(10) Pub. No.: US 2014/0080795 A1
(43) Pub. Date: Mar. 20, 2014

(54) NRF2 Deficiency Influences Susceptibility to Steroid Resistance via HDAC2 Reduction

(71) Applicant: Irfan Rahman, Rochester, NY (US)

(72) Inventor: Irfan Rahman, Rochester, NY (US)

(73) Assignee: University of Rochester, Rochester, NY (US)

(21) Appl. No.: 13/988,038

(22) PCT Filed: Nov. 18, 2011
(86) PCT No.: PCT/US11/61380

§ 371 Date: Dec. 3, 2013

Related U.S. Application Data
(60) Provisional application No. 61/415,591, filed on Nov. 19, 2010.

(51) Int. Cl. A61K 31/7088; A61K 31/26; A61K 31/58

(52) U.S. Cl. CPC A61K 31/7088; A61K 31/58; A61K 31/26
USPC 514/171; 514/514; 435/375; 435/455; 514/44 R

(57) ABSTRACT

Methods for the treatment or prevention of diseases which are caused by the decreased concentrations of histone deacetylase 2 (HDAC2) in cells are described. The diseases which may be treated by the methods of the invention include chronic obstructive pulmonary disease (COPD) and asthma, including steroid resistant COPD and asthma. The invention provides methods for treating or preventing of diseases caused by the degradation of HDAC2 by providing to the subject in need of treatment or prevention a molecular compound capable of preventing the degradation of HDAC2. Such molecular compounds include Nuclear factor (erythroid-derived 2)-like 2 (Nrf2) activators. Methods are further provided for the treatment and prevention of COPD and asthma by providing to a subject in need of such treatment and prevention a nucleic acid which causes expression of HDAC2 in lung cells and/or a nucleic acid which causes expression of Nrf2 in lung cells.

### STATEMENT OF GOVERNMENT SUPPORT

This invention was made with U.S. Government support under NIH Grant Numbers IR01HL085613, 1 IR01HL097751, IR01HL092842, awarded by the National Institutes of Health. The U.S. Government has certain rights in the invention.

The present invention relates to methods of treating inflammatory diseases, including steroid resistant inflammatory diseases, by modulating the levels and/or activity of Nuclear factor (erythroid-derived 2)-like 2 (Nrf2, also known as NFE2L2).

The present invention further relates to methods of treating inflammatory diseases by modulating levels and/or activity of histone deacetylase 2 (HDAC2) through either direct modulation or modulation of factors which affect HDAC2 levels and/or activity, such as Nuclear factor (erythroid-derived 2)-like 2 (Nrf2, also known as NFE2L2), as well as overcoming the steroid resistance.

### BACKGROUND OF THE INVENTION

Chronic obstructive pulmonary diseases (COPD) are conditions of the lungs, including emphysema and chronic bronchitis, in which the airways of the lungs are narrowed, making it difficult to breath. While exposures to other air pollutants, such as fumes from biomass fuel and industrial air pollutants can cause COPD, the primary cause of the disease is exposure to cigarette smoke. The inflammation that leads to COPD has been associated with the expression of proinflammatory genes driven by the Nuclear Factor KB (NF-KB) transcription factor complex (Ito. et al, N Engl J Med, 352: 1967-1976 (2005)). There is no cure for COPD, but subjects are often treated with corticosteroids to help slow the progression of the disease. The long term treatment of COPD with corticosteroids often leads to steroid resistance, whereby the treatment becomes progressively less effective.

Cigarette smoke (CS) is a complex mixture of oxidants/free radicals and different chemical compounds which include reactive aldehydes and quinones known to cause oxidative stress in the lungs (Pryor, et al., Ann N Y Acad Sci, 686: 12-27 (1993)), is the primary risk factor for the pathogenesis of COPD(Rahman, et al., Eur Respir J, 28:219-242 (2006); Rahman, et al., Am J Respir Crit Care Med, 166:490-495 (2002)). It is believed that cigarette smoking is the primary etiological factor in steroid resistance observed in subjects with COPD and asthmatics who smoke (Barnes, Am J Respir Crit Care Med, 161 :342-344 (2000)), a phenomenon which is also observed in other inflammatory diseases, such as rheumatoid arthritis (Chikanza, et al., Rheumatology (Oxford), 43:1337-1345 (2004)) and inflammatory bowel disease (Farrell, et al. J Endocrinol, 178:339-346 (2003)). On ligand binding, corticosteroids suppress inflammation by glucocorticoid receptor recruitment of histone deacetylase 2 (HD AC2) specifically to acetylated histones on promoters of proinflammatory genes such as IL-8 and GM-CSF (Ito, et al. Mol Cell Biol, 20:6891-6903 (2000)).

Histone deacetylases (HDACs) are a family of cellular enzymes that regulate gene expression by catalyzing the removal of acetyl groups from the lysine tails of core histones (de Ruijter, et al., Biochem J, 370:737-749 (2003)). HDAC2 is a class I histone deacetylase that resides almost exclusively in the nucleus and is a critical part of co-repressor complexes recruited to proinflammatory gene promoters by associated proteins (de Ruijter, et al., Biochem J, 370:737-749 (2003)). The inability of corticosteroids to recruit HDAC2 or the presence of post-translationally modified HDAC2 may explain the abnormal inflammatory response and ineffectiveness of corticosteroid therapy in subjects with COPD (Ito, et al., Faseb J, 15:1 110-11 12 (2001); Marwfck, et al., Am J Respir Cell MoI Biol, 31:633-642 (2004); Yang, et al., Am J Physiol Lung Cell MoI Physiol, 291:L46-57 (2006)). In animal exposure experiments, lungs of rats and different strains of mice exposed to cigarette smoke (CS) exhibit significantly decreased HDAC2 expression and activity similar to that observed in peripheral blood mononuclear cells (PBMCs) of subjects with mild to severe asthma, alveolar macrophages of COPD subjects and chronic asthmatics who smoke (Ito, et al., Faseb J, 15:1 110-11 12 (2001); Marwfck, et al., Am J Respir Cell MoI Biol, 31 :633-642 (2004); Yang, et al., Am J Physiol Lung Cell MoI Physiol, 291 :L46-57 (2006); Cosio, et al., Am J Respir Grit Care Med, 170:141-147 (2004); Yao, et al., Am J Physiol Lung Cell MoI Physiol, 294:L1 174-1 186 (2008)). Bronchial biopsies of COPD subjects and asthmatics who smoke exhibit decreased HDAC2 expression which correlates with disease severity, increased cytokine production and corticosteroid insensitivity (Ito. et al, N Engl J Med, 352: 1967-1976 (2005); Ito, et al., Am J Respir Crit Care Med, 166:392-396 (2002)). Several studies have suggested that corticosteroid insensitivity is closely related to reduced expression of HDAC2. Increased post-translational modification and subsequently degradation of HDAC2 required to balance increased IKKα-mediated chromatin modification (acetylation of histone proteins) of proinflammatory promoters in lungs in response to CS (Yang, et al., Am J Respir Cell MoI Biol, 38:689-698 (2008); Rajendrasozhan, et al, Antioxid Redox Signal, 10:799-811 (2008)). In view of this, it has been recently shown that HDAC2 is modified by nitration of tyrosine residues, formation of protein-aldehyde adducts in response to CS, or by reactive aldehydes subsequently leading to loss of HDAC2 activity (Marwfck, et al., Am J Respir Cell MoI Biol, 31:633-642 (2004); Yang, et al., Am J Physiol Lung Cell MoI Physiol, 291:L46-57 (2006)). Despite overwhelming evidence that loss of HDAC2 by oxidant-mediated post-translational modification is closely linked to corticosteroid insensitivity, the molecular mechanisms of CS-induced degradation of HDAC2, are still unclear.

Nuclear factor (erythroid-derived 2)-like 2 (Nrf2) regulates the cellular antioxidant response by upregulating genes involved in augmenting cellular antioxidant capacity and by inducing the genes that detoxify reactive oxygen species or electrophilic compounds (Itoh, et al., Biochem Biophys Res Commun, 236:313-322 (1997); Jaiswal, et al., Free Radic Biol Med, 36:1199-1207 (2004)). The level of Nrf2 are decreased in conditions of oxidative stress (Goven, et al., Thorax 63:916-924 (2008); Malhotra, et al., Am J Respir Crit Care Med, 178:592-604 (2008); Garbin, et al., PLoS One 4:e8225-e8236 (2009)), which accounts for persistent and abnormal inflammation due to imbalance of oxidants/antioxidants in the lung (Garbin, et al., PLoS One 4:e8225-e8236 (2009); Harju, et al., Am J Respir Crit Care Med, 166:754-759 (2002); Tomaki, et al., Pulm Pharmacol Ther, 20:596-605 (2007)). Mice lacking Nrf2 gene are susceptible to cigarette smoke (CS)-induced pulmonary inflammation and emphysema (Rangasamy, et al., J Clin Invest, 114:1248-1259 (2004)). Hence, it is possible that abnormal inflammation seen in these mice is oxidant-dependent and resistance to steroids perhaps due to oxidant-mediated reduction in HDAC2 levels.

The present invention provides novel and facile methods for overcoming corticosteroid resistance in the treatment of COPD and other corticosteroid resistant disorders through the targeting and manipulation of HDAC and Nrf2.

### SUMMARY OF THE INVENTION

The present invention provides methods and compositions for the treatment of inflammatory disorders, including corticosteroid resistant inflammatory disorders. Inflammatory disorders which may be treated using the methods and compositions of the present invention include chronic obstructive pulmonary disorder (COPD), acne vulgaris, asthma, autoimmune diseases, celiac disease, chronic prostatitis, glomerulonephritis, hypersensitivities, inflammatory bowel diseases, pelvic inflammatory disease, reperfusion injury, rheumatoid arthritis, sarcoidosis, transplant rejection, vasculitis, and interstitial cystitis.

It is an object of the present invention to provide methods for the treatment and prevention of diseases related to reduced cellular levels of histone deacetylase 2 (HDAC2) in a subject. The diseases which may be treated or prevented by the methods of the present invention are caused by reduced cellular levels of HDAC2, which can lead to the expression of genes involved in inflammation which are driven by the NF-κB promoter. The diseases which may be treated by the methods of the present invention include chronic obstructive pulmonary disease (COPD) and asthma, along with other inflammatory diseases.

It is an object of the present invention to provide methods for the treatment and prevention of COPD and asthma in a subject by providing to the subject a nucleic acid sequence which causes expression of HDAC2, nuclear factor (erythroid-derived 2)-like 2 (Nrf2), or both HDAC2 and Nrf2 in the lung cells of the subject.

It is a further object of the present invention to provide methods for the treatment and prevention of diseases related to reduced cellular levels of HDAC2 in a subject by providing to the subject a molecular compound which is capable of maintaining cellular levels of HDAC2. Such molecular compounds include Nrf2 activators.

It is a further object of the present invention to provide methods for the treatment of an inflammatory disorder in a subject comprising concomitantly administering to the subject a pharmaceutical formulation comprising an effective amount of a corticosteroid and an effective amount of a Nrf2 activator.

In one embodiment, the pharmaceutical formulation further comprises a phosphodiesterase 4 (PDE4) inhibitor. In another embodiment, the pharmaceutical formulation further comprises an antioxidant.

It is another object of the present invention to provide methods for the treatment of a corticosteroid resistant inflammatory disorder in a subject comprising concomitantly administering to the subject: a pharmaceutical formulation comprising an amount of corticosteroid to which the inflammatory disorder is resistant and a pharmaceutical formulation comprising an effective amount of a Nrf2 activator; wherein the Nrf2 activator causes the amount of corticosteroid to be effective in treating the inflammatory disorder. In certain embodiments, the corticosteroid and the Nrf2 activator may be in the same formulation.

In one embodiment, the method for treatment of a corticosteroid resistant inflammatory disorder further comprises concomitant administration of a PDE4 inhibitor. In another embodiment, the method for treatment of a corticosteroid resistant inflammatory disorder further comprises concomitant administration of an antioxidant. The PDE4 inhibitor and antioxidant, when provided, may be in the same pharmaceutical formulation as corticosteroid or Nrf2 activator or may be provide in a separate pharmaceutical formulation.

It is a further object of the invention to provide pharmaceutical formulations comprising an effective amount of corticosteroid and an effective amount of a Nrf2 activator. In certain embodiments, the pharmaceutical formulations of the invention may further comprise a PDE4 inhibitor. In certain other embodiments, the pharmaceutical formulations may further comprise an antioxidant.

In one embodiment, the pharmaceutical formulations of the invention are inhalable pharmaceutical formulations. In another embodiment, the pharmaceutical formulations of the invention are solid oral dosage forms. In yet another embodiment, the pharmaceutical formulations of the invention are injectable pharmaceutical formulations.

It is a further object of the present invention to provide methods for increasing cellular levels of HDAC2 in a cell comprising, providing the cell and contacting the cell with an Nrf2 activator, wherein the Nrf2 activator causes an increase in the cellular levels of HDAC2. In certain embodiments, the methods are performed in vivo. In certain other embodiments, the methods are performed in vitro. In certain other embodiments, the methods are performed in vitro. In still other embodiments, the methods are performed ex vivo.

It is a further object of the present invention to provide methods for increasing cellular levels of HDAC2 in a cell comprising, providing the cell, and contacting the cell with a nucleic acid that causes expression of an Nrf2 polypeptide in the cell, wherein the expression of the Nrf2 polypeptide causes an increase in the cellular levels of HDAC2. In certain embodiments, the methods are performed in vivo. In certain other embodiments, the methods are performed in vitro. In still other embodiments, the methods are performed ex vivo. In certain embodiments, the nucleic acid comprises a nucleic acid sequence encoding a polypeptide which is at least 80% identical to SEQ ID NO: 2. In other embodiments, the nucleic acid comprises a nucleic acid sequence encoding a polypeptide which is at least 90% identical to SEQ ID NO:2. In still other embodiments, the nucleic acid comprises a nucleic acid sequence encoding a polypeptide which is at least 95% identical to SEQ ID NO: 2.

It is a further object of the invention to provide pharmaceutical formulations for the treatment of corticosteroid resistant inflammatory disorders comprising an effective amount of corticosteroid and an effective amount of a Nrf2 activator. In certain embodiments, the pharmaceutical formulations of the invention may further comprise a PDE4 inhibitor. In certain other embodiments, the pharmaceutical formulations may further comprise an antioxidant.

It is a further object of the present invention to provide the use of Nrf2 activators for the treatment of corticosteroid resistant inflammatory disorders. The Nrf2 activators may be administered in a pharmaceutical formulation and may be administered concomitantly with a corticosteroid. In one embodiment, use of Nrf2 activators for the treatment of corticosteroid resistant inflammatory disorders comprises concomitant administration of a PDE4 inhibitor. In another embodiment, use of Nrf2 activators for the treatment of corticosteroid resistant inflammatory disorders comprises concomitant administration of an antioxidant.

It is a further object of the present invention to provide the use of Nrf2 activators for the treatment of inflammatory disorders associated with low cellular levels of HDAC2. The Nrf2 activators may be administered in a pharmaceutical formulation and may be administered concomitantly with a corticosteroid. Inflammatory disorders associated with low cellular levels of HDAC2 may be corticosteroid resistant inflammatory disorders. In one embodiment, use of Nrf2 activators for the treatment of inflammatory disorders associated with low cellular levels of HDAC2 comprises concomitant administration of a PDE4 inhibitor. In another embodiment, use of Nrf2 activators for the treatment of inflammatory disorders associated with low cellular levels of HDAC2 comprises concomitant administration of an antioxidant.

### DESCRIPTION OF THE DRAWINGS

FIG. 1 shows experimental results demonstrating that HDAC2−/− mice are more susceptible to CS-induced lung inflammation. WT and HDAC2−/− mice were exposed to filtered air or acute CS for 3 days. Mice were sacrificed at 24 h post-last exposure, and lungs were lavaged. Bronchoalveolar lavage cells were prepared on cytospin slides and stained with Diff-Quik. (A) Total number of neutrophils in BALF. (B) MCP-1 levels were measured in mouse lung tissue by ELISA. Data are shown as mean±SEM (n=4 to 6). *P<0.05, **P<0.01, ***P<0.001 significant compared with corresponding air groups. #P<0.05, and ###P<0.001 compared with corresponding WT mice exposed to CS. sP<0.05, compared with air-exposed WT mice.

FIG. 2 shows experimental results demonstrating that HDAC2−/− mice are less responsive to budesonide in response to LPS-induced lung inflammation. (A) WT mice were exposed to saline or aerosolized LPS (1 mg/ml). Nuclear extracts from mouse lungs were separated on SDS-PAGE gel, and HDAC2 protein levels were determined by immunoblotting. (B) Band intensity of HDAC2 levels normalized to actin. (C and D) WT and HDAC2−/− mice were pretreated with budesonide (3 mg/kg body weight) for 3 days at 1 h prior to aerosolization of LPS. (C) Neutrophils in BALF were determined following differential staining on cytospin slides. (D) MCP-1 levels were measured from lung homogenates of mouse lung tissue by ELISA. Data are shown as mean±SEM (n=4 to 6). ***P<0.001, significant compared with corresponding controls as denoted in figure. Sal: Saline; Bud: Budesonide.

FIG. 3 shows experimental results demonstrating that increased lung inflammation is associated with HDAC2 reduction in Nrf2−/− mice exposed to CS. (A) WT and Nrf2−/− mice were exposed to CS for 3 days. Differential counts from BALF were determined by Diff-Quik staining of cytospin slides. (B) Immunoprecipitated HDAC2 from lungs of naïve WT and Nrf2−/− mice were analyzed for deacetylase activity using specific HDAC deacetylase activity kit. (C) WT and Nrf2−/− mice were exposed to filtered air or whole body CS for 3 days. Lung tissue nuclear extracts were analyzed for HDAC2 relative expression. (B) HDAC2 levels normalized to actin expression. Data are shown as mean±SEM (n=4 to 9). *P<0.05, **P<0.01, and ***P<0.001, significant compared with corresponding air-exposed controls. #P<0.05 and ###P<0.001, significant compared with CS-exposed WT controls. $$$P<0.001, compared with air-exposed WT mice.

FIG. 4 shows experimental results demonstrating that Nrf2−/− mice are unresponsive to budesonide following LPS exposure. Nrf2−/− mice were treated with budesonide (1 or 3 mg/kg body weight) by intranasal administration followed by LPS exposure. (A) Percentage of neutrophils in BALF was determined following differential staining on cytospin slides. (B) MCP-1 levels were measured in lung tissue homogenates by ELISA. Data are shown as mean±SEM (n=4 to 6). ***P<0.001 significant compared with corresponding controls. Sal: Saline; Bud: Budesonide.

FIG. 5 shows experimental results demonstrating that HDAC2 ablation leads to spontaneous airspace enlargement (emphysema) in mice. Unlavaged left lung samples from 4 and 6 months old WT and 2, 4, 10, 14 and 18 months old HDAC2 −/− were inflated with 1% agarose and paraffin embedded. Sections were stained with hematoxylin and eosin (H & E).

FIG. 6 shows a plot demonstrating that Alveolar airspace enlargement was significantly higher in HDAC2−/− mice compared to WT. Data are shown as mean±SEM (n=3-4). # # # P<0.001, significant compared with 4 months WT. $ $ $ P<0.001, significant compared with 6 months WT.

FIG. 7 shows images of gels showing that MMP9 activity in mouse lungs was determined using enzyme zymography assay.

FIG. 8 shows results of experiments in which pro-inflammatory MCP-1 and KC levels were measured from lung homogenates of HDAC2−/− mice by ELISA. Data are shown as mean±SEM (n=4 to 6 mice). ***P<0.001 # # # P<0.001 significant compared with respective controls as denoted in figure. Sal: Saline; Bud: Budesonide.

FIG. 9 shows results of experiments in which pro-inflammatory MCP-1 and KC levels were measured from lung homogenates of Nrf2 −/− mice by ELISA. Data are shown as mean±SEM (n=4 to 6 mice). **P<0.01, and ***P<0.001 significant compared with respective controls. # P<0.05 compared with LPS+budesonide high dose versus saline+budesonide high dose.

### DESCRIPTION OF THE INVENTION

The present invention relates to methods for the treatment of disorders resistant to treatment by corticosteroids by providing agents which increase the activity of Nuclear factor (erythroid-derived 2)-like 2 (Nrf2) or by increasing cellular levels of Nrf2 in conjunction with administration of corticosteroids. The methods of the present invention may involve the regulation of the production of genes involved in inflammation by controlling the cellular levels or activity of histone deacetylase 2 (HDAC2). The methods of the present invention may be carried out by directly affecting HDAC2 or by affecting other upstream factors that subsequently lead to a change in the cellular levels or activity of HDAC2, such as Nrf2.

The present invention further relates to methods for the regulation of the production of genes involved in inflammation by controlling the cellular levels or activity of histone deacetylase 2 (HDAC2). The methods of the present invention may be carried out by directly affecting HDAC2 or by affecting other upstream factors that subsequently lead to a change in the cellular levels or activity of HDAC2.

### Definitions

To facilitate an understanding of the present invention, a number of terms and phrases are defined below.

The term “cellular levels of Nrf2” as used herein may refer to the intracellular concentration of Nrf2. Cellular levels of Nrf2 may therefore be modified by causing an increase in production of the Nrf2 protein or by preventing the degradation of Nrf2 protein already present in the cell. The cellular level may also be increased functionally by causing more Nrf2 protein to be directed to a certain cellular compartment, such as the nucleus. In these cases, the total level of Nrf2 in the cell may not change, but the concentration of Nrf2 in the specific cellular compartment may be increased.

The term “cellular levels of HDAC2” as used herein may refer to the intracellular concentration of HDAC2. Cellular levels of HDAC2 may therefore be modified by causing and increase in production of the HDAC2 protein or by preventing the degradation of HDAC2 protein already present in the cell. The cellular level may also be increased functionally by causing more HDAC2 protein to be directed to a certain cellular compartment, such as the nucleus. In these cases, the total level of HDAC2 in the cell may not change, but the concentration of HDAC2 in the specific cellular compartment may be increased.

The term “activity of Nrf2” as used herein relates to the activity of Nrf2 proteins in inducing transcription by binding to an antioxidant-response element, leading to an increased rate of transcription activity of genes regulated by Nrf2.

The terms “Nrf2 activator” and “Nuclear factor (erythroid-derived 2)-like 2 activator” as used herein relate to chemical compounds or elements that increase the activity of Nrf2.

The term “activity of HDAC2” as used herein relates to the rate by which an HDAC2 protein catalyzes the deacetylation of histones and non-histone proteins. The activity of an HDAC2 proteins may be increased though the contacting of the HDAC2 proteins with various compounds, such as co-factors and activators, which lead to an increased rate of catalysis for HDAC2.

The terms “inflammatory disorder,” or “inflammatory disease” may refer to a group of disorders including chronic obstructive pulmonary disorder (COPD), acne vulgaris, asthma, autoimmune diseases, celiac disease, chronic prostatitis, glomerulonephritis, hypersensitivities, inflammatory bowel diseases, pelvic inflammatory disease, reperfusion injury, rheumatoid arthritis, sarcoidosis, transplant rejection, vasculitis, and interstitial cystitis.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

Administration “in combination with” one or more further therapeutic agents includes simultaneous (concurrent) and consecutive administration in any order.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

An “effective amount” of an agent as disclosed herein is an amount sufficient to carry out a specifically stated purpose. An “effective amount” can be determined empirically and in a routine manner, in relation to the stated purpose.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. If present, modification to the nucleotide structure can be imparted before or after assembly of the polymer. The sequence of nucleotides can be interrupted by non-nucleotide components. A polynucleotide can be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars can be replaced, for example, by phosphonate groups, phosphate groups, protected by standard protecting groups, or activated to prepare additional linkages to additional nucleotides, or can be conjugated to solid supports. The 5′ and 3′ terminal OH can be phosphorylated or substituted with amines or organic capping group moieties of from 1 to 20 carbon atoms. Other hydroxyls can also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages can be replaced by alternative linking groups. These alternative linking groups include, but are not limited to, embodiments wherein phosphate is replaced by P(O)S (“thioate”), P(S)S (“dithioate”), “(O)NR2 (”amidate“), P(O)R, P(O)OR′, CO or CH2 (”formacetal“), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “vector” means a construct, which is capable of delivering, and optionally expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. One such non-limiting example of a sequence alignment algorithm is the algorithm described in Karlin et al, 1990, Proc. Natl. Acad. Sci., 87:2264-2268, as modified in Karlin et al., 1993, Proc. Natl. Acad. Sci., 90:5873-5877, and incorporated into the NBLAST and)(BLAST programs (Altschul et al., 1991, Nucleic Acids Res., 25:3389-3402). In certain embodiments, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, Calif.) or Megalign (DNASTAR) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48):444-453 (1970)) can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be longer than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2: 482 489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. Identity can exist over a region of the sequences that is at least about 10, about 20, about 40-60 residues in length or any integral value there between, and can be over a longer region than 60-80 residues, for example, at least about 90-100 residues, and in some embodiments, the sequences are substantially identical over the full length of the sequences being compared, such as the coding region of a nucleotide sequence for example.

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of and/or “consisting essentially of are also provided.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### Nrf2 Activators

The methods of the present invention are based, in part, on the discovery by the inventor that cellular HDAC2 levels are decreased in animals lacking Nrf2 and that animals lacking HDAC2 are more susceptible to cigarette smoke induced lung inflammation, as is shown in the Examples below. Without wishing to be bound by theory, the decreased levels of HDAC2 are thought to then lead to increased transcription of genes involved in inflammation through NF-κB driven promoters. The resulting inflammation leads to the development and progression of COPD and other inflammatory disorders. In particular, loss of HDAC2 activity is thought to be a primary factor in the development of corticosteroid resistance in the treatment of diseases associated with inflammation The determination of this regulation of HDAC2 provides for several novel points of control of inflammation for the treatment of COPD and other diseases for which inflammation is controlled by HDAC2. Further description of the interaction of HDAC2, Nrf2 and other factors is given in the Examples below.

In certain embodiments, the methods of the present invention are used in the treatment of corticosteroid resistant inflammatory disorders, such as corticosteroid resistant COPD. For the purposes of the present invention, corticosteroid resistant inflammatory disorders include inflammatory disorders that were initially responsive to corticosteroid treatment but became resistant as well as disorders that were initially resistant to corticosteroid treatment. A corticosteroid resistant inflammatory disorder may be completely resistant to the corticosteroid, e.g., the corticosteroid has little to no effect on the disorder, or an inflammatory disorder may be corticosteroid resistant even if the corticosteroid continues to have some reduced effect treating the disorder. The methods of the present invention may completely or partially alleviate corticosteroid resistance. These methods may make the inflammatory disorder as responsive to corticosteroid treatment as it originally was, or may improve the responsiveness of the disorder to some fraction of its original responsiveness. It is further contemplated that the methods of the present invention cause the inflammatory disorder to be more responsive to the corticosteroid than it originally was.

Improvements in corticosteroid resistance can be measured using methods well known in the art along with diagnosis by medical personnel. For example, determining whether corticosteroid resistance is improving in COPD or asthma patients can involve the use of diagnostic tests known in the art for COPD and asthma, including spirometry tests, arterial blood gas tests, bronchial provocation tests, exercise tolerance tests, exhaled nitric oxide tests, x-rays, x-ray computed tomography, bronchioscopy, mucus culture and pH probe studies. Determining whether corticosteroid resistance is improving in rheumatoid arthritis can involve the use of tests known in the art such as blood tests such as erythrocyte sedimentation rate, C-reactive protein tests and rheumatoid factor tests, x-rays, magnetic resonance imaging (MRI) and ultrasound. Determining whether corticosteroid resistance is improving in inflammatory bowel disease can involve the use of tests known in the art such as blood tests, x-ray, MRI, colonoscopy, and intestinal biopsy.

In certain other embodiments, the methods of the present invention may be used for the treatment and/or prevention of COPD and other inflammatory disorders, whether corticosteroid resistant or not. As the methods of the present invention may reduce or eliminate the underlying cause of COPD, namely inflammation, the methods of the present invention can be preventative. If the methods of the present invention are begun while a subject is exposed to factors related to COPD development, such as cigarette smoke, but before the subject shows clinical signs of the disease (shortness of breath, wheezing, rapid breathing, coughing, etc.), the methods may be useful in preventing the clinical signs of the disease from ever manifesting or may slow the manifestation of the disease. The methods may also be used in treatment of COPD and other diseases once clinical signs have manifested. As the inflammation which is controlled by the present methods is necessary for the progression of COPD, the methods of the present invention may be used to slow or reverse the progression of the disease. The methods of the present invention may be able to reverse the progression of the disease to the point where the clinical symptoms are absent, whereby the subject will be considered to be cured or in remission.

There are various Nrf2 activators known in the art which are suitable for use in the present invention, including tert-butylhydroquinone (tBHQ), sulforaphane, Oltipraz (4-methyl-5-(2-pyrazinyl)-3-dithiolethione), bardoxolone methyl (also known as CDDO-Me or RTA 402) from Reata pharmaceuticals, dihydro-CDDO-trifluoroethyl amide (dh404), resveratrol, anethole dithiolethione, 6-methylsulphinylhexyl isothiocyanate, curcumin, caffeic acid phenethyl ester, and 4′-bromoflavone, 1,2,3,4,6-Penta-O-Galloyl-Beta-D-Glucose, 1,2-Diphenol (Catechol), 1,2-Dithiole-3-Thione, 1,4-Diphenols (P-Hydroquinone), 1-[2-Cyano-3-,12-Dioxooleana-1,9(11)-Dien-28-Oyljlmidazole (CDDO-Imidazol), 15-Deoxy-12,14-Pgj2,1-Chloro-2,4-Dinitrobenzene, 2,3,7,8-Tetrachlorodibenzo-P-Dioxin, 2-Cyano-3,12-Dioxooleana-1,9(11)-Dien-28-Oic Acid (CDDO), 2-Indol-3-Yl-Methylenequinuclidin-3-Ols, 3-Hydroxyanthranilic Acid, 3-Methylcholanthrene, 4-Hydroxyestradiol, 4-Hydroxynonenal, 6-Methylsulfinylhexyl, Isothiocyanate, 9-Cis-Retinoic Acid, Acetaminophen, Acetylcarnitine, Acrolein, Allyl Isothiocyanate, Alpha-Lipoic Acid, Apomorphine, Arsenic, AUR ((2,3,4,6-Tetra-O)-Acetyl-1-Thio-D-Glucopyranosato-S)(Triethylphosphine) Gold(I), Autg ((1-Thio-D-Glucopyranosato) Gold(I), Autm (Sodium Aurothiomalate), Bis(2-Hydroxybenzylidene)Acetone, Bleomycin, B-Naphthoflavone, Broccoli Seeds, Bucillamine, Butein, Butylated Hydroxyanisole, Butylated Hydroxytoulene, Cadmuim Chloride, Cafestol, Carbon Monoxide, Carnosol, Catechol, chalcones (1,3-Diphenyl-2-propen-1-ones), chalcone derivatives (such as those described in Kumar, et al., J Med Chem, 54:4147-59 (2011) and Yang et al., Free Rad Biol Med, 51:2073-2081(2011), the disclosures of each of which are hereby incorporated by reference herein), Chlorogenic Acid, Cigarette Smoke, Cobalt (Cobalt Chloride), Copper, Coumarin, Curcumin, Deprenyl (Selegiline), Dexamethasone 21-Mesylate, Diallyl Disulfide, Diallyl Sulfide, Diallyl Trisulfide (DATS), Diesel Exhaust, Diethylmaleate, Epicatechin-3-Gallate, Epigallocatechin-3-Gallate, Eriodictyo, Ferulic Acid (Trans-4-Methoxycinnamic Acid, 99% Purity), Fisetin, Flunarizine, Gallic Acid (3,4,5-Trihydroxybenzoic Acid), Gentisic Acid, Glucose Oxidase, Glycosides From Digitalis Purpurea, Heme, Hemin, Hydrogen Peroxide, Hyerpoxia, Indole-3-Carbinol, Indomethacin, Insulin, Iodoacetic Acid, Kahweol Palmitate, Laminar Flow, Lead, Limettin (LMTN), Lipoic Acid, Lipopolysacharide, Luteolin, Lycopene, Menadione, Mercury, Nickel (II), Nitric Oxide-Donating Aspirin, Oxidized Low-Density Lipoproteins, Paraquat, Parthenolide, P-Coumaric Acid (Trans-4-Hydroxycinnamic Acid), Phenethyl Isothiocyanate, Phloretin Phorbol 12-Myristate 13-Acetate (PMA), P-Hydroxybenzoic Acid, Proteasome Inhibitor MG-132, Proteasome Inhibitors (Lactacystin Or MG-132), Pyrrolidine Dithiocarbamate, Quercetin, Quercetin 3-O-Beta-L-Arabinopyranoside, Sodium Arsenite, Spermidine, Spermine, Spermine Nonoate, TNF-Alpha, Trans-Stilbene Oxide, Triterpenoid-155, Triterpenoid-156, Triterpenoid-162, Triterpenoid-225, Tunicamycin, Ultraviolet A, Irradiation, Wasabi Extract, Xanthohumol (XH), Zerumbone, Zinc, Patulin, Methosyvone, Dehydrovariabilin, Biochanin A, Pdodfilox, 8-2′-Dimethoxyflavone, 6,3′-Dimethoxyflavone, Pinosylvin, Gentian Violet, Gramicidin, Thimerosal, Cantharidin, Fenbendazole, Mebendazole, Triacetylresveratrol, Resveratrol, Tetrachloroisopthalonitrile, Simvastatin, Valdecoxib, beta-Peltatin, 4,6-Dimethoxy-5-methylsioflavone, Nocodazole, Pyrazinecarboxamide, (±)-thero-l-Phenyl-2-decanoylamino-3-morpholino-l-propanol hydrochloride, SU4132. Additional examples of Nrf2 activators can be found in U.S. Published Patent Application 2011/0250300 to Biswal et al. and U.S Published Patent Application 2004/0002463 to Honda et al., the disclosures of each of which are hereby incorporated by reference herein.

Also included among useful Nrf2 activators are pharmaceutically acceptable molecular conjugates or salt forms of the activators described above, that maintain activity as Nrf2 activators as defined herein. Examples of pharmaceutically acceptable salts of Nrf2 activators include sulfate, chloride, carbonate, bicarbonate, nitrate, gluconate, fumarate, maleate, or succinate salts. Other embodiments of pharmaceutically acceptable salts contain cations, such as sodium, potassium, magnesium, calcium, ammonium, or the like. Other embodiments of useful Nrf2 activators are hydrochloride salts. For providing enhanced cell permeability to a Nrf2 activator moiety, various conjugated forms are useful, e.g., Nrf2 activator-lipid conjugates, emulsified conjugates of Nrf2 activators, lipophillic conjugates of Nrf2 activators, and liposome- or micelle-conjugated Nrf2 activators. (Fenske, D B et al., Biochim Biophys Acta, 2001:1512(2):259-72; Khopade, A J et al., Drug Deliv. 2000: 7(2): 105-12; Lambert, D M et al., Eur. J. Pharm. Sci. 2000: 11 Suppl 2:S15-27; Pignatello, R et al., Eur J Pharm Sci. 2000: 10(3):237-45; Allen, C et al., Drug Deliv. 2000: 7(3):139-45; Dass, C R et al., Drug Deliv. 2002: 9(1): 11-8; Dass, C R, Drug Deliv. 2000:7(3): 161-82; which are hereby incorporated by reference herein).

The Nrf2 activators can be synthesized by known chemical means or can be procured commercially.

In certain embodiments, the methods of the present invention contemplate the administration of an Nrf2 activator concomitantly with a corticosteroid. Corticosteroids contemplated by the present invention include: hydrocortisone type corticosteroids such as hydrocortisone, hydrocortisone acetate, cortisone acetate, tixocortol pivalate, prednisolone, methylprednisolone, and prednisone; acetonides such as triamcinolone acetonide, triamcinolone alcohol, mometasone, amcinonide, budesonide, desonide, fluocinonide, fluocinolone acetonide, and halcinonide; betamethasone type corticosteroids such as betamethasone, betamethasone sodium phosphate, dexamethasone, dexamethasone sodium phosphate, and fluocortolone; corticosteroid esters such as hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, hydrocortisone-17-butyrate, 17-aceponate, 17-buteprate, and prednicarbate. In certain embodiments, inhaled corticosteroids such as flunisolide, fluticasone propionate, triamcinolone acetonide, beclomethasone dipropionate and budesonide are preferred. Examples of commercially available inhalable corticosteroid formulations for use in the methods of the present invention include AEROBID® (flunisolide), AEROSPAN® (flunisolide), FLOVENT® (fluticasone), FLOVENT ROTADISK® (fluticasone), PLUMICORT® (budesonide), PLUMICORT TURBUHALER® (budesonide) and ADVAIR® (fluticasone and salmeterol).

In certain embodiments, the methods of the present invention contemplate the administration of an Nrf2 activator concomitantly with a phosphodiesterase type 4 (PDE4) inhibitor and optionally a corticosteroid. PDE4 inhibitors contemplated by the present invention include mesembrine, rolipram, ibudilast, piclamilast, luteolin, roflumilast (DAXAS®), cilomilast, and diazepam.

In certain other embodiments, the methods of the present invention contemplate the administration of an Nrf2 activator concomitantly with an antioxidant and optionally a corticosteroid. Antioxidants contemplated by the present invention include N-acetylcysteine, N-acystelyn, thio-N-acetylcysteine, N-isobutyrylcysteine, glutathione, glutathione esters, S-carboxymthylcysteine (Carbocysteine), erdostein, fudosteine, thioredoxin, procysteine, ergothioneine, ascorbic acid, lipoic acid, uric acid, beta-carotene, retinol, melatonin, alpha-tocopherol, ubiquinol, salen compounds, M40419, M40403, M40419, manganese-metaloporphyrins (such as AEOL-10113 and AEOL10150), metaloporphyrins (MnTRAP), MnTE-2-Pyp, EUK compounds (such as EUK-189), edselen, BXT-51072 (ALT-2074), BXT-51077, edaravone (MC-186), lazaroids/tirilazad (such as U75412E or ttirilazad mesylate), nitrone spin traps (NXY-059, STANZ), peroxynitrite decomposition catalysts (FeTMPyp, FP15), porphyrins, glutaredoxins, peroxiredoxins, polyphenols, flavonoids, curcumin, resveratrol, quercetin, sulphoraphane, lycopene, Acai, Apocynin, vitamin D and omega-3-fatty acid.

The present invention contemplates the concomitant administration of an Nrf2 activator with the additional agents discussed above. Concomitant administration may include the administration of an Nrf2 activator in the same pharmaceutical formulation with one of these additional agents, e.g., the administration of a pharmaceutical formulation containing an Nrf2 activator and one or more additional agents. Concomitant administration may further include administration of a pharmaceutical formulation containing an Nrf2 activator at the same time as the administration of a separate pharmaceutical formulation containing an additional agent. Concomitant administration also includes the administration of an Nrf2 activator during the course of therapy with an additional agent, even if the Nrf2 activator and the additional agent are not administered at the same time of the day.

Pharmaceutical formulations of the invention contain the Nrf2 activator and, optionally, contain pharmaceutically acceptable solvent(s), adjuvant(s) and/or pharmaceutically acceptable non-medicinal, non-toxic carrier(s), binder(s), thickener(s), and/or filler substance(s) that are known to the skilled artisan for the formulation of tablets, pellets, capsules, solutions, emulsions, suspensions, and any other form suitable for use. The carriers which can be used include glucose, lactose, sucrose, gum acacia, gelatin, mannitol, starch, starch paste, magnesium trisilicate, talc, corn starch, keratin, colloidal silica, potato starch, urea, medium chain length triglycerides, dextrans, petrolatum, and other carriers suitable for use in manufacturing preparations, in solid, semisolid, or liquid form. In addition auxiliary, stabilizing, thickening and coloring agents and perfumes can be used. Also contemplated are additional medicinal or nutritive additives in combination with at least one Nrf2 activator, such as vitamins and the like.

The pharmaceutical formulations containing the Nrf2 activator may administered by any suitable method, including by oral, parenteral, inhalation and transdermal routes. Such methods of administering are well known to those of skill in the art.

The pharmaceutically formulations can be formulated for oral or enteral administration, for example, as tablets, troches, caplets, microspheres, hard or soft capsules, lozenges, aqueous or oily suspensions, dispersible powders or granules, emulsions, syrups, elixirs or enteral formulas. In certain embodiments, solid oral dosage forms, such as tablets or caplets are used.

Controlled release or continuous dosing regimens are also useful. The pharmaceutical industry has developed many types of slow and/or sustained-release technology. Sustained -release formulations employ several methods. The most common is a tablet containing an insoluble core; a drug applied to the outside layer is released soon after the medication is ingested, but drug trapped inside the core is released more slowly. Capsules containing multiparticulate units of drug with coatings that dissolve at different rates are designed to give a sustained-release effect.

The Nrf2 activators of the present invention may also be formulated for administration by inhalation, as is well known in the art. The Nrf2 activators may be formulated as are inhalable corticosteroids, such as in aerosol form, containing a propellant such as HFA-134a (1,1,1,2-tetrafluoro ethane) or may be formulated with excipients such as lactose for inhalation. Examples of inhalable corticosteroids having suitable systems for administration by inhalation include AEROBID®, AEROSPAN®, FLOVENT®, FLOVENT ROTODISK®, PLUMICORT®, and PLUMICORT TURBUH ALER®. The Nrf2 activators of the present invention may be formulated with a corticosteroid, for co-administration, or, alternatively, the Nrf2 activators may be formulated alone using an inhalation system similar to those used for inhalable corticosteroids. Further examples of inhalable formulations include those in which the active ingredient is entrapped in a liposome, such as those systems described in U.S. Pat. No. 5,192,528 to Kishore, which is hereby incorporated by reference herein.

In accordance with the invention, pharmaceutically acceptable compositions are formulated to deliver an effective amount of at least one Nrf2 activator by the above-described or any other pharmaceutically acceptable systemic delivery system, preferably in an amount of about 10 to about 100 milligrams per kilogram of body mass per dose of Nrf2 activator, more preferably about 20 to about 80 milligrams per kilogram of body mass per dose, and most preferably about 25 to about 50 milligrams per kilogram of body mass per dose. It is contemplated that one to two doses of the Nrf2 activator may be delivered to the subject each day, although two to four or more daily doses are also in accordance with the present invention. The useful pharmaceutically acceptable composition can be formulated and manufactured at more than one concentration unit of Nrf2 activator, such that modular incremental amounts of Nrf2 activators are easily administered to subjects of various sizes as needed.

In certain embodiments, injectable pharmaceutical formulations are used. In accordance with the inventive method, administration by injection can be in a bolus or by infusion over a period of one to thirty minutes, and most preferably during a period of one to about fifteen minutes. If by infusion, the practitioner skilled in the art is also cautious in regulating the total infusion volume, rate of liquid infusion, and electrolyte balance to avoid adverse physiological effects related to these.

For example, for delivery by intravascular infusion or bolus injection into a subject, the Nrf2 activator is preferably in a solution that is suitably balanced, osmotically (e.g., about 0.15 M saline) and with respect to pH, typically between pH 7.2 and 7.5; preferably the solution further comprises a buffer, such as a phosphate buffer (e.g., in a phosphate buffered saline solution). The solution is formulated to deliver a dose of about 10 to 100 milligrams of Nrf2 activator per kilogram body mass in a pharmaceutically acceptable fluid volume over a maximum of about thirty minutes.

In certain embodiments, pharmaceutical formulations to be administered may contain more than one Nrf2 activator. In other embodiments, separate pharmaceutical formulations containing different Nrf2 activators may be administered during the course of treatment.

In embodiments of the present invention where one or more additional agents are not formulated in the same formulation as the Nrf2 activator, the additional agents may be formulated as is well known in the art or formulated as described above. Effective amounts for the above agents are well known in the art. For example the effective amounts for commercially available corticosteroids can be found on the product labeling. It is also contemplated that small dosages of corticosteroids, PDE4 inhibitors or antioxidants may be effective in concomitance with an Nrf2 activator than when any of these additional agents are used alone.

### Nrt2 and HDAC Upregulation

In other embodiments of the present invention, methods are provided for treating COPD and other inflammatory disorders through the upregulation of Nrf2 expression. In certain embodiments, it may be desired to upregulate Nrf2 expression in a the cells of a subject that poorly express Nrf2 or express Nrf2 in a less functional form.

In other embodiments of the invention, methods are provided whereby HDAC2 is provided to a subject in order to increase or maintain the level of HDAC2 in the cell. In further embodiments of the invention, methods are provided whereby Nrf2 is provided to a subject in order to increase of maintain the level of HDAC2 and Nrf2 in the cell. In still further embodiments of the invention, methods are provided whereby both HDAC2 and Nrf2 are provided to a subject in order to increase of maintain the level of HDAC2 and Nrf2 in the cell. Such methods can be implemented using standard gene therapy methods as are well known in the art. The methods known in the art can be viral or non-viral. Examples of gene therapy methods applicable to the methods of the present invention can be found in Phillips, Gene Therapy Methods, Methods in Enzymology vol. 346, Elsevier Science & Technology Books, 2002.

In preferred embodiments of the invention, the HDAC2 and/or Nrf2 genes are delivered directly to the lungs. Recently, methods have been developed in the art by which genes have been delivered directly to the lungs using aerosol-mediated gene delivery. These methods for gene delivery to the lungs should be adaptable for the delivery of HDAC2 and/or Nrf2 to the lungs. Examples of such methods can be found in Hwang, et al., Respiratory and Critical Care Medicine, 2009: 179: 1131-1140, and Vachani, et al., “Gene therapy for mesothelioma and lung cancer,” Am J Respir Cell Mol Biol. 2010 Apr; 42(4):385-93, which are hereby incorporated by reference herein. The methods for delivering genes to lungs known in the art may be modified by a person of skill in the art to provide the Nrf2 and/or HDAC2 genes in place of the genes provided in these known methods.

Vectors for the delivery of the genes of the present invention include adenovirus vectors (Bachtarzi, et al., Expert Opin. Drug Deliv., 5:1231-1240 (2008); Douglas, et al., Mol. Biotechnol., 36:71-80 (2007)), retrovirus vectors, lentivirus vectors (Breckpot et al., Curr. Gene Ther. 8:438-448 (2008); Schambach, et al., Curr. Gene Ther., 8:474-482 (2008)), adeno-associated virus vectors (Daya, et al., Clin. Microbiol. Rev. 21:583-593 (2008); Schultz, et al., Mol. Ther., 16:1189-1199 (2008)), vaccinia/fowl pox vectors (Garber, et al., PLos One, 4:e5445 (2009); Kirn, et al., Nat. Rev. Cancer, 9:64-71 (2009)) and nonviral vectors (Chesnoy, et al., Annu. Rev. Biophys. Biomol. Struct. 29:27-47 (2000); Niidome, et al., Gene Ther. 9:1647-1652 (2002)), the cited references for each of which are hereby incorporated by reference herein.

The HDAC2 gene to be delivered in the methods of the present invention may by a nucleic acid encoding the full length amino acid sequence of HDAC2, as is shown in SEQ ID NO: 1, or may encode smaller functional version of HDAC2. It is also contemplated that the HDAC2 gene to be delivered may encode an amino acid sequence that is not 100% identical to SEQ ID NO:1, such as a sequence that is 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:1.

The Nrf2 gene to be delivered in the methods of the present invention may by a nucleic acid encoding the full length amino acid sequence of Nrf2, as is shown in SEQ ID NO: 2, or may encode smaller functional version of Nrf2. It is also contemplated that the Nrf2 gene to be delivered may encode an amino acid sequence that is not 100% identical to SEQ ID NO:2, such as a sequence that is 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:2.

The above methods describe embodiments of the invention, and it should be apparent to one of skill in the art that there are other methods, which are not expressly described herein, which fall within the scope of the invention. The attached Examples provided below are for illustrative purposes, and are not meant to limit the scope of the claims in any way.

### EXAMPLES

### Example 1

Inhaled corticosteroids remain the therapy of choice for inflammatory diseases, such as asthma and chronic obstructive pulmonary disease (COPD). However, inhaled or systemic corticosteroids fail to attenuate chronic inflammation in these patients due to increased oxidative stress (Keatings, et al., Am J Respir Crit Care Med, 155:542-548 (1997); Hew, et al., Am J Respir Crit Care Med, 174:134-141 (2006); Rahman, et al., Eur Respir J, 28:219-242 (2006)). Corticosteroids suppress inflammation by recruiting histone deacetylase 2 (HDAC2) to NF-κB-driven pro-inflammatory gene promoters thereby inhibiting the transcription of these genes (Ito, et al. Mol Cell Biol, 20:6891-6903 (2000)). Thus, the loss of corticosteroid ability to suppress inflammation in COPD and asthma may be due to the loss of HDAC2 protein (Barnes, et al., Lancet, 373:1905-1917 (2009)). However, the mechanism for steroid resistance via HDAC2 reduction in oxidative stress-prone condition in vivo is not known.

Nuclear factor (erythroid-derived 2)-like 2 (Nrf2) regulates the cellular antioxidant response by upregulating genes involved in augmenting cellular antioxidant capacity and by inducing the genes that detoxify reactive oxygen species or electrophilic compounds (Itoh, et al., Biochem Biophys Res Commun, 236:313-322 (1997); Jaiswal, et al., Free Radic Biol Med, 36:1199-1207 (2004)). The level of Nrf2 are decreased in conditions of oxidative stress (Goven, et al., Thorax 63:916-924 (2008); Malhotra, et al., Am J Respir Crit Care Med, 178:592-604 (2008); Garbin, et al., PLoS One 4:e8225-e8236 (2009)), which accounts for persistent and abnormal inflammation due to imbalance of oxidants/antioxidants in the lung (Garbin, et al., PLoS One 4:e8225-e8236 (2009); Harju, et al., Am J Respir Crit Care Med, 166:754-759 (2002); Tomaki, et al., Pulm Pharmacol Ther, 20:596-605 (2007)). Mice lacking Nrf2 gene are susceptible to cigarette smoke (CS)-induced pulmonary inflammation and emphysema (Rangasamy, et al., J Clin Invest, 114:1248-1259 (2004)). Hence, it is possible that abnormal inflammation seen in these mice is oxidant-dependent and resistance to steroids perhaps due to oxidant-mediated reduction in HDAC2 levels.

It was hypothesized that Nrf2 regulates steroid sensitivity via HDAC2 in response to inflammation in mouse lung. Furthermore, HDAC2 deficiency leads to steroid resistance in attenuation of lung inflammatory response triggered by increased oxidative stress. This hypothesis was tested in mice lacking Nrf2 and HDAC2 to determine the ability of corticosteroids to inhibit lung inflammatory response induced by CS and LPS.

### Materials and Methods

### Materials

Unless otherwise stated, all biochemical reagents used in this study were purchased from Sigma (St. Louis, Mo.). HDAC2 antibody was obtained from Santa Cruz Biotechnology (Santa Cruz, Calif.). Monoclonal β-actin antibody was obtained from Calbiochem (La Jolla, Calif.).

### Mice

The Nrf2 knockout (KO) mouse strain (Nrf2−/− on C57BL/6J background) used in this study is described earlier (Barnes, et al., Lancet, 373:1905-1917 (2009)) and was generously supplied by Prof. Masayuki Yamamoto, University of Tsukuba, Japan via the RIKEN BioResource Center, Tsukuba, Japan. HDAC2 mutant (HDAC2−/− on C57BL/6J background) mice were kindly provided by Dr. J.A. Epstein (University of Pennsylvania School of Medicine, Philadelphia, Pa.) (Trivedi, et al., Nat Med 13:324-331 (2007)). These mice express a truncated and catalytically inactive form of HDAC2 with exons 9-14 replaced by a LacZ fusion gene created via a gene-trap method (Trivedi, et al., Nat Med 13:324-331 (2007)). Wild-type (WT) C57BL/6J mice were purchased from Jackson laboratories (Bar Harbor, Me.). All animal protocols for this study were approved by the University Committee on Animal Research of the University of Rochester.

### Cigarette Smoke and Lipopolysacharide (LPS) Exposure to Mice

Eight to ten week-old adult wild-type (WT), Nrf2−/− and HDAC2−/− were exposed to diluted mainstream CS generated from 3R4F filtered research grade cigarettes as described previously (Yang, et al., Am J Respir Cell Mol Biol, 38:689-698 (2008); Yao, et al., Am J Physiol Lung Cell Mol Physiol, 294:L1174-1186 (2008)) for 1 h twice daily with 1 h interval in between for 3 days using a Baumgartner-Jaeger CSM2072i cigarette smoke-generating machine (CH Technologies, Westwood, N.J.). Smoke concentration and airflow were adjusted to obtain a constant particulate matter concentration of 300 mg/m3 total particulate matter (TPM) (Yang, et al., Am J Respir Cell Mol Biol, 38:689-698 (2008); Yao, et al., Am J Physiol Lung Cell Mal Physiol, 294:L1174-1186 (2008)). Mice exposed to filtered air served as controls. Mice were sacrificed at 24 h post-last CS exposure.

LPS exposure studies in mice were performed as described previously (Yao, et al., Am J Respir Cell Mol Biol, 39:7-18 (2008)). Briefly, age-matched WT, Nrf2−/− and HDAC2−/− were exposed to aerosolized Escherichia coli LPS (1 mg/ml) for 8 minutes. Aerosolized saline-exposed mice were used as controls, and animals were sacrificed at 24 h post last exposure.

### Corticosteroid Treatment

Budesonide in dry-powered form was dissolved in 70% ethanol and then diluted with saline prior to administration. Twenty-five microliters of budesonide solution, corresponding to 1 or 3 mg/kg body weight, was administered via an intranasal route to each mouse for 3 days followed by LPS exposure at 1 h after last budesonide treatment (Southam, et al., Eur Respir J, 32:970-978 (2008)).

### Bronchoalveolar Lavage

Mice were anaesthetized by pentobarbital (Abbott Laboratories, Abbott Park, Ill.) intraperitoneal injection (100 mg/kg body weight) before sacrifice. Lungs were then removed and lavaged three times with 0.6 ml of 0.9% sodium chloride with cannula inserted into the trachea. Total lavage fluid for each mouse was combined and then centrifuged. Supernatants were frozen at −80° C. until required for further analysis and cell pellets resuspended in 1 ml of saline, and total number of cells determined using a haemocytometer. Differential counts (minimum 500 per slide) were determined using Diff-Quik (Dade Behring, Newark, Del.)-stained cytospin slides.

### Lung Tissue Protein Extraction

Cytoplasmic and nuclear proteins were extracted from frozen lung tissue samples as described (Adenuga, et al., Am J Respir Cell Mol Biol, 40:464-473 (2009)). Whole cell lysate was extracted from lung tissue after homogenization in RIPA buffer (Yang, et al., Am J Respir Cell Mol Biol, 38:689-698 (2008); Yao, et al., Am J Physiol Lung Cell Mol Physiol, 294:L1174-1186 (2008)).

### Immunoblotting

Protein estimation was performed by the bicinchoninic acid (BCA) method as described by the manufacturer (Fierce, Rockford, Ill.). Mouse lung nuclear extracts (10 μg-20 μg) were electrophoresed on 7.5% SDS-polyacrylamide gels, electro-blotted on PVDF membranes (Millipore, Burlington, Mass.). Membranes were incubated with primary antibody in a 1:1000 dilution in 5% BSA in TBS. After extensive washing, primary antibodies were detected with secondary antibodies linked to horse-radish peroxidase (Dako, Carpinteria, Calif.) and bound complex detected with enhanced chemiluminescence (PerkinElmer, Waltham, Mass.).

### Cytokine Analysis

Monocyte chemotactic protein 1 (MCP-1) levels were measured from mouse lung soluble protein by enzyme-linked immunosorbent assay (ELISA) with the duo-antibody kit (R & D Systems). Results were normalized to protein concentration per sample.

### HDAC2 Activity Assay

HDAC2 was immunoprecipitated from lung homogenates (500 μg protein) by incubating overnight with anti-HDAC2 antibody (2 μg). Beads were washed and incubated with Color de Lys substrate (Biomol) for 80 minutes with rocking at 37° C. 30 μL aliquots from each sample were placed in 96-well plates and HDAC specific buffer added. Color de Lys developer was then added and incubated for a further 20 minutes with rocking at 37° C. Color development was monitored at 405 nm. HDAC2 activity was expressed relative to standard curve generated from 0-500 μM Color de Lys deacetylated standard.

### Statistical Analysis

Data expressed as mean±SEM. Statistical significance was calculated using one-way Analysis of Variance (ANOVA) with STATVIEW software. NIH ImageJ software was used for densitometry analysis. P<0.05 as significant compared to relative controls.

### Results

### HDAC2−/− Mice are More Susceptible to CS-Induced Lung Inflammation

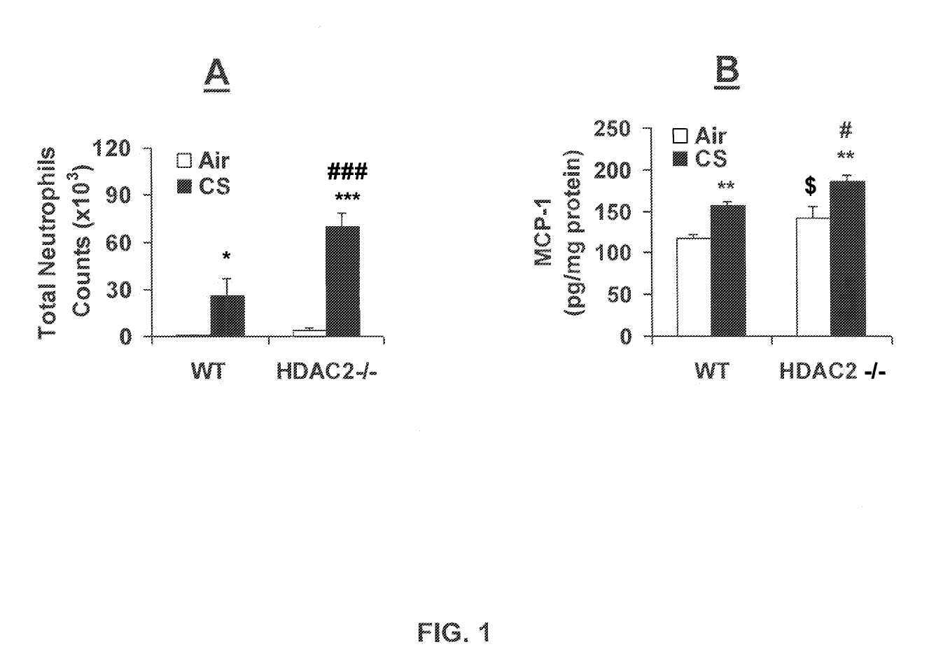

CS exposure induced neutrophil influx in bronchoalveolar lavage fluid (BALF) of WT mice, which was significantly increased in HDAC2−/− mice (FIG. 1A). Consistent with this finding, air- and CS-exposed HDAC2−/− mice showed significant increase in MCP-1, a monocyte chemoattractant (FIG. 1B); and KC, a neutrophil chemoattractant (data not shown), release in the lungs as compared to corresponding air- and CS-exposed WT mice, suggesting the susceptibility of HDAC2 deficient mice to CS-induced lung inflammation.

### HDAC2−/− Mice are Less Responsive to Budesonide in Inhibiting LPS-Induced Lung Inflammation

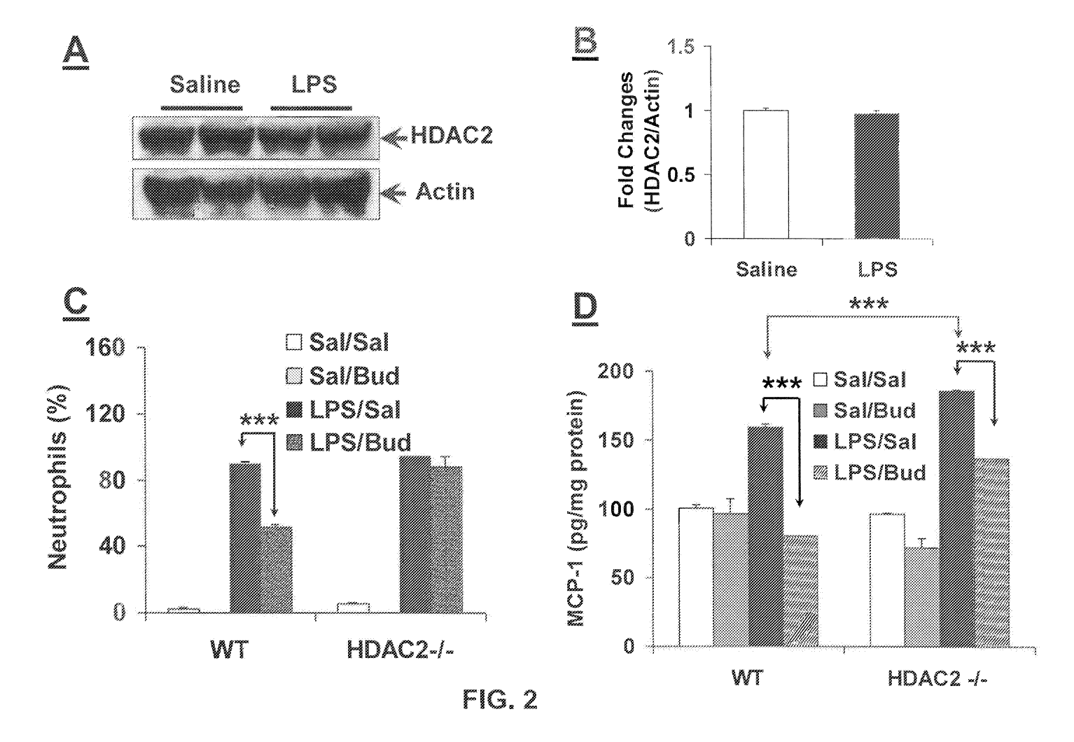

LPS was used as a different lung inflammation-triggering agent rather than CS since HDAC2 is inactivated and degraded in response to CS exposure both in mouse lungs in vivo, and in epithelial cells and macrophages in vitro (Adenuga, et al., Am J Respir Cell Mol Biol, 40:464-473 (2009); Marwick, et al., Am J Respir Cell Mol Biol, 31:633-642 (2004);] Yang, et al., Am J Physiol Lung Cell Mol Physiol, 291:L46-57 (2006); Adenuga, et al., Arch Biochem Biophys, 498:62-73 (2010)), whereas LPS aerosolization in WT mice had no effect on HDAC2 protein levels in mouse lung (FIGS. 2A and B). Not surprisingly, neutrophil counts in BALF were significantly reduced in budesonide-pretreated LPS-exposed WT mice as compared to saline-pretreated LPS-exposed WT mice (FIG. 2C).

Budesonide pretreatment had no effect on neutrophil counts in BALF of HDAC2−/− mice (FIG. 2C). However, pretreatment of budesonide significantly lowered the MCP-1 (FIG. 2D) and KC (data not shown) release in WT mouse lung in response to LPS exposure. The levels of MCP-1 in lungs of HDAC2−/− mice were reduced by budesonide in response to LPS exposure (FIG. 2D). However, the efficacy of budesonide in decreasing MCP-1 release in HDAC2−/− mice (26.4%) was lower than that in WT mice (49.4%, P<0.01). Budesonide, however, had no appreciable effect on KC release in response to LPS exposure in HDAC2−/− mice (data not shown). Consistent with the MCP-1 and KC data, matrix metalloproteinase 9 (MMP9) activity was significantly elevated in HDAC2−/− mouse lungs compared to WT lungs with only a minimal effect of budesonide treatment observed (data not shown). Overall, these data suggest that HDAC2 deficiency leads to steroid insensitivity in terms of attenuating lung inflammatory response.

Enhanced Lung Inflammation is Associated with HDAC2 Reduction in Nrf2−/− Mice Exposed to CS

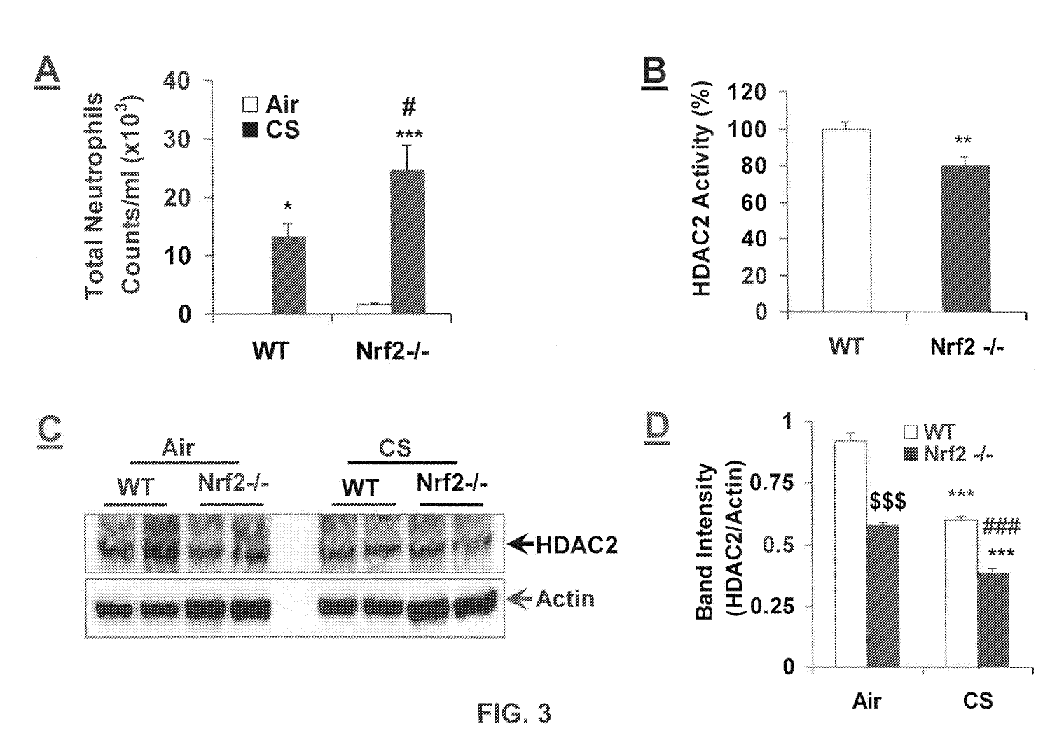

It was determined whether mice deficient in the antioxidant transcription factor, Nrf2, would be more susceptible to CS-mediated oxidative stress to cause lung inflammation Neutrophil influx, as a marker for increased vascular permeability and pulmonary inflammation, was increased in lungs of Nrf2−/− mice as compared to WT mice in response to 3 days CS exposure (FIG. 3A).

HDAC2 protein level and deacetylase activity are significantly decreased in response to CS-mediated oxidative stress in mouse lungs (Adenuga, et al., Am J Respir Cell Mol Biol, 40:464-473 (2009)), therefore, we speculated that HDAC2 protein levels in lung may be altered in Nrf2−/− mice, which are susceptible to oxidative stress. Naïve Nrf2−/− mouse lung showed decreased HDAC2 activity compared to WT mouse lungs (FIG. 3B). To determine if this difference was due to accelerated loss of HDAC2 protein in lungs of Nrf2−/− mice, HDAC2 protein abundance was compared in nuclear extracts of Nrf2−/− and WT mice. Consistent with reduction in HDAC2 activity, the HDAC2 protein level was also significantly decreased in lung nuclear extracts of Nrf2−/− mice as compared to WT mice in the absence of CS exposure (FIGS. 3C and D). Three days of CS exposure further decreased the levels of HDAC2 protein in Nrf2−/− mice compared to WT mice (FIGS. 3C and D). These results suggest that the loss of HDAC2 in Nrf2 deficient mice is a crucial component of increased susceptibility to oxidative stress-induced inflammatory response in the lungs.

### Nrf2−/− Mice are Not Responsive to Budesonide in Inhibiting LPS-Induced Lung Inflammation

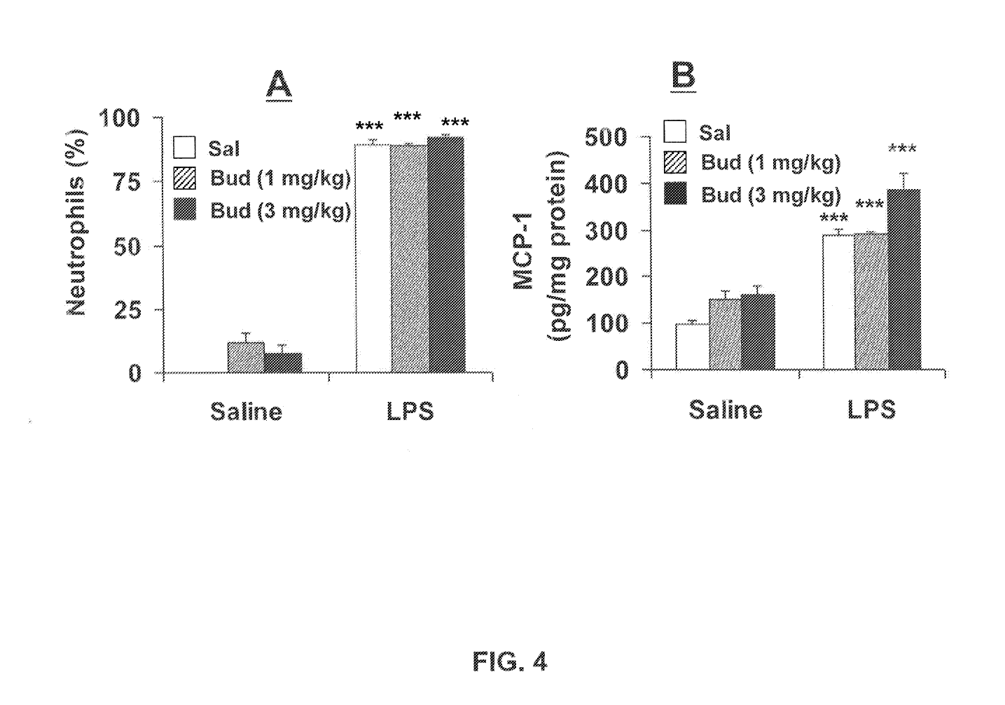

Decreased HDAC2 protein levels and deacetylase activity in naïve Nrf2−/− mice suggested that these mice may also exhibit resistance to steroid-mediated attenuation of lung inflammation. As shown in FIG. 2C, budesonide (3 mg/kg) pretreatment attenuated lung neutrophil influx following LPS exposure by ˜50% in WT mice. However, the neutrophils influx in BALF was not altered by pretreatment of budesonide (1 and 3 mg/kg) in Nrf2−/− mice in response to LPS exposure (FIG. 4A). Similarly, pretreatment of budesonide did not reduce LPS-induced release of MCP-1 (FIG. 4B) and KC (data not shown) release in Nrf2−/− mouse lung. These results suggest that the inability of steroids to inhibit lung inflammation possibly be due to HDAC2 reduction in lungs of Nrf2 deficient mice.

### Discussion

Although HDAC2 has been implicated in increased susceptibility to inflammation and steroid resistance in vitro (Yang, et al., Am J Physiol Lung Cell Mol Physiol, 291:L46-57 (2006); Adenuga, et al., Arch Biochem Biophys, 498:62-73 (2010); Ito, et al., FASEB J 15:1110-1112 (2001)), no such studies are available to determine the role of HDAC2 in vivo in lung in response to inhaled toxicants. In this study, mice expressing mutant HDAC2 (Trivedi, et al., Nat Med 13:324-331 (2007); Zimmermann, et al., Cancer Res, 67:9047-9054 (2007); Kramer, Trends Pharmacol Sci, 30:647-655 (2009)) were used to study the steroid resistance in response to CS exposure. HDAC2−/− mice showed increased neutrophil recruitment in the lung in response to CS as compared to WT mice exposed to CS. Neutrophil recruitment was correlated strongly with the release of KC and MCP-1 in lung tissue suggesting the susceptibility of these mice to augmentation of CS-induced inflammation.

Since CS induces degradation of other HDACs (HDAC1 and HDAC3) that may be crucial for inflammation (Yang, et al., Am J Physiol Lung Cell Mol Physiol, 291:L46-57 (2006)), it was important to determine a specific role for HDAC2 in steroid resistance utilizing an exposure model with no effect on HDAC levels or activity. Hence, it was hypothesized that HDAC2−/− mice would exhibit a poor response to budesonide in response to LPS exposure. As expected, budesonide had no significant effect in reducing KC release in HDAC2−/− mice lungs in response to LPS. Furthermore, budesonide partially blocked MCP-1 release in the lungs of HDAC2−/− mice versus complete inhibition of MCP-1 in WT mice. Budesonide was also ineffective in reducing MMP9 activity in the mutant mice. Thus, these data suggest that basal HDAC2 deficiency leads to increased inflammatory response in the lung which is further augmented by LPS exposure. Furthermore, steroids have poor efficacy in HDAC2-ablated mice in response to pro-inflammatory challenge in the lung.

Increased susceptibility to oxidative stress (or deficiency of antioxidants) may exhibit phenotypes similar to HDAC2−/− mice in terms of steroid resistance. Nrf2−/− mice, which are susceptible to oxidative stress/cigarette smoke, were used to study the steroid resistance in controlling the lung inflammatory response. Disruption of Nrf2 enhanced susceptibility to acute CS-induced neutrophilic inflammation which is consistent with an earlier report (Rangasamy, et al., J Clin Invest, 114:1248-1259 (2004)).

Surprisingly, in air-exposed Nrf2−/− mice, neutrophil infiltration and RelA/p65 phosphorylation on ser276 and ser536 (unpublished data) were markedly enhanced in the lungs. It is possible that loss of Nrf2 leads to activation of NF-κB by increasing the pool of CREB-binding protein (CBP) available to interact with RelA/p65 (Liu, et al., Biochim Biophys Acta, 1783:713-727 (2008); Sun, et al., Mol Cell Biol 29:2658-2672 (2009)), and thus causing induction of NF-B-dependent pro-inflammatory genes. The data provided suggest that Nrf2 deficiency led to increased lung neutrophil influx and basal activation of NF-κB, which are augmented by CS exposure.

The finding that loss of Nrf2 induces an oxidative-stress phenotype in the lungs led to the hypothesis that increased oxidative stress status of the lung in Nrf2−/− mice would induce a reduction in HDAC2 activity and level. Lungs from naïve Nrf2−/− mice showed marked reduction in HDAC2 abundance and subsequently reduced HDAC2 deacetylase activity compared to naïve WT mice. Consistent with these data, exposure of Nrf2−/− mice to CS rapidly accelerated the rate of HDAC2 degradation suggesting the oxidant burden in lungs of Nrf2−/− is a key factor in loss of HDAC2 with or without exposure to environmental toxicants. The reduction in levels of HDAC2 in Nrf2−/− mice alternatively suggests that ARE-mediated Nrf2 activation may be required for HDAC2 transcription. However, the molecular regulation of HDAC2 expression or transcription factor(s) binding site is not clearly known (Kramer, Trends Pharmacol Sci, 30:647-655 (2009)). It may also be possible that nuclear Nrf2 stabilizes the HDAC2 co-repressor complex on pro-inflammatory genes, whereas deficiency of Nrf2 leads to degradation of HDAC2 and activation of NF-κB-CBP binding on pro-inflammatory promoters (Liu, et al., Biochim Biophys Acta, 1783:713-727 (2008); Sun, et al., Mol Cell Biol 29:2658-2672 (2009)). Preliminary data support this contention showing activation of CBP in lungs of Nrf2 null mice (unpublished data).

It was next determined whether the increased inflammatory response and decreased HDAC2 protein levels in Nrf2−/− mice render them resistant to steroids in inhibiting the inflammatory response. WT and Nrf2−/− mice were exposed to aerosolized LPS following intranasal budesonide treatment. LPS induced severe neutrophilic inflammation in mouse lungs (unlike macrophage influx by CS) with no subsequent reduction in HDAC2 protein expression. Pre-treatment of WT mice with a high dose of budesonide significantly blocked LPS-induced lung neutrophilic influx which is consistent with budesonide-mediated inhibition of lung tissue KC levels. However, LPS-exposed Nrf2−/− mice failed to respond to both low and high dose budesonide treatments in blocking neutrophil influx, attenuating KC and MCP-1 release, and inhibiting RelA/p65 nuclear accumulation (unpublished data) in the lung. The observation of steroid resistance in Nrf2−/− and HDAC2−/− mice with the finding that HDAC2 protein levels are diminished in Nrf2−/− mice suggests a strong in vivo linkage between HDAC2 abundance and steroid sensitivity via oxidative stress.

In conclusion, loss of HDAC2 is a critical factor in inhaled toxicant-mediated lung inflammation especially in regulating the anti-inflammatory effects of glucocorticoids in mouse lungs. Oxidative stress-susceptible Nrf2−/− mice showed reduced HDAC2 levels and deacetylase activity in lungs, and are susceptible to CS- and LPS-induced inflammation. Interestingly, the loss of Nrf2 potentially leads to steroid resistance due to HDAC2 reduction. It can be speculated that the high oxidant status in the lung of Nrf2−/− mice leads to inactivation of endogenous HDAC2 through post-translational modifications, such as nitrosylation of tyrosine residues and carbonylation of cysteine, histidine and lysine residues. This may then block the ability of ligand-bound glucocorticoid receptors to recruit active HDAC2 to promoters of pro-inflammatory genes. Nevertheless, the rapid loss of HDAC2 and NF-κB activation in CS-exposed Nrf2−/− mice suggest steroid resistance is clearly a multifactorial cascade of events that perhaps start with oxidant/antioxidant imbalance and then precipitates at a much more rapid decline in HDAC2. This may have implications for devising better therapies for patients who are refractory/insensitive to steroid treatments, such as patients with COPD, asthma, rheumatoid arthritis, and inflammatory bowel disease (Barnes, et al., Lancet, 373:1905-1917 (2009)).

### Example 2

### Methods

### Mice

The Nrf2 knockout (Nrf2−/−) mouse strain (C57BL/6J background) used in this study has been described previously (Itoh et al., Biochem Biophys Res Commun, 236: 313-322 (1997)) and was generously supplied by Prof. Masayuki Yamamoto, University of Tsukuba, Japan via the RIKEN BioResource Center, Tsukuba, Japan. HDAC2 mutant mice (HDAC2−/−) on a C57BL/6J background were kindly provided by Dr. J. A. Epstein (University of Pennsylvania School of Medicine, Philadelphia, Pa.) (Trivedi et al., Nat Med, 13: 324-331 (2007)). Wild-type C57BL/6J mice were purchased from Jackson laboratories (Bar Harbor, Me.). Mouse breeding was carried out under the supervision of the vivarium facility of the University of Rochester and all standards and protocols for this study were approved by the University Committee on Animal Research.

### Cigarette Smoke Exposure to Mice

8-10 week-old wild type, Nrf2−/− and HDAC2−/− were exposed whole body to filtered air (FA) or dilute, mainstream CS from 3R4F filtered research grade cigarettes (11.7 mg total particulate matter yield per cigarette; University of Kentucky Tobacco Research and Development Centre [UKTRDC], Lexington, Ky.) (Li et al., Toxicol Sci, 111: 247-253 (2009); Yang et al., Am J Respir Cell Mol Biol, 38: 689-698 (2008); Yao et al., Am J Physiol Lung Cell Mol Physiol, 294: L1174-1186 (2008); Yao H, et al., Am J Pathol, 172: 1222-1237 (2008)) for 1 h twice daily with another 1 h interval in between for 3 days using a Baumgartner-Jaeger CSM2072i cigarette smoke-generating machine (CH Technologies, Westwood, N.J.) according to the Federal Trade Commission protocol (1 puff/min of 2-s duration and 35-ml volume). Mice exposed to filtered air served as controls. Smoke concentration and airflow were adjusted to obtain a constant particulate matter exposure of 300 mg/m3 total particulate matter (TPM). Mice were sacrificed at 2 or 24 h post last exposure.

LPS exposure studies were performed as described (Yao et al., Am J Respir Cell Mol Biol 39: 7-18 (2008)). Briefly, age-matched wild-type, Nrf2−/− and HDAC2−/− were exposed to aerosolized Escherichia coli LPS diluted to 1 mg/ml for 8 minutes. Aerosolized saline-exposed mice were used as controls and animals were sacrificed at 24 h post last exposure (Yao et al., Am J Respir Cell Mol Biol 39: 7-18 (2008)).

### Corticosteroid Treatment

Budesonide (Sigma, St Louis, Mo.) in dry-powered form was dissolved in 70% ethanol and then diluted with saline prior to exposure. Twenty-five microliters of budesonide solution, corresponding to 1 or 3 mg/kg body weight, was administered by an intranasal route to each mouse for 3 days followed by LPS exposure 1 h after last budesonide treatment (Southam et al., 2008).

### Lung Tissue Protein Extraction for Cytokine Analysis

Cytoplasmic and nuclear proteins were extracted from frozen lung tissue samples as described (Adenuga et al., Am J Respir Cell Mol Biol 40: 464-473 (2009)). Briefly, approximately 150 mg of lung tissue was homogenized in 0.5 ml of buffer A (10 mM HEPES (pH 7.8), 10 mM KCl, 2 mM MgCl2, 1 mM dithiothreitol, 0.1 M EDTA, 0.2 mM NaF, 0.2 mM Na orthovandate, 1% (v/v) Nonidet P-40, 0.4 mM phenylmethyl sulfonyl fluoride, and 1 μg/ml leupeptin) on ice for 30 minutes. Homogenates were briefly centrifuged 4° C. to remove cellular debris. Cytoplasmic extracts were collected after centrifuging supernatants for 30 seconds at 13,000 g at 4° C. Pellet was resuspended in 50 μl of buffer C (50 mM HEPES (pH 7.8), 50 mM KCl, 300 mM NaCl, 0.1 M EDTA, 1 mM dithiothreitol, 10% (v/v) glycerol, 0.2 mM NaF, 0.2 mM Na orthovanadate, and 0.6 mM phenylmethylsulfonyl fluoride) and placed on the rotator in the cold room for 30 minutes, centrifuged at 13,000 g in an Eppendorf tube for 5 minutes and supernatant collected as the nuclear extract and kept frozen at −80° C. Whole cell lysate was extracted from lung tissue after homogenization in RIPA buffer (Yang et al., Am J Respir Cell Mol Biol, 38: 689-698 (2008); Yao H, et al., Am J Pathol, 172: 1222-1237 (2008)).

### Histological Analysis

Histological analysis of mouse lungs was performed as described (Yao et al., Am J Respir Cell Mol Biol 39: 7-18 (2008)). Briefly, fresh, unlavaged mouse structure was preserved by inflating with 1% low melt agarose at a pressure of 25 cm H2O. Lungs were then fixed 4% neutral buffered paraformaldehyde (PFA), embedded in paraffin for sectioning. Tissue sections were stained with hematoxylin and eosin (H&E) and the mean linear intercept (Lm) of airspace was determined (Yao et al., Am J Respir Cell Mol Biol 39: 7-18 (2008)).

### Cytokine Analysis

Monocyte chemotactic protein 1 (MCP-1) levels were measured from mouse lung soluble protein by enzyme-linked immunosorbent assay (ELISA) with the MCP-1 duo-antibody kit (R & D Systems). Results were normalized to protein concentration per sample.

### Zymography for MMP9 Activity Assay

Lung tissues were homogenised in 400 μl lysis buffer (50 mM Tris-HCl, pH 7.4, with protease inhibitors) on ice. 100 μg protein was then mixed with equal volume sample buffer (80 mmol Tris-HCl, pH 6.8, 4% SDS, 10% glycerol, 0.01% bromophenol blue) and then loaded on a 7.5% SDS-poly acrylamide gel containing 1 mg/ml gelatin. After electrophoresis, gels were rinsed in distilled water, washed three times for 15 minutes each in 150 ml 2.5% Triton X-100 solution. Gels were then incubated in 100-150 ml of 50 mmol/l Tris-HCl (pH 7.5), 10 mmol/l CaCl₂, 1 μM ZnCl2, 1% Triton X-100 and 0.02% NaN₃. After incubation, gels were stained with 100 ml Coomassie blue R-250 (Sigma) for 3 h and then destained 1 h with destaining solution (50% methanol, 10% acetic acid). Gels were washed in distilled water for 20 minutes and then scanned.

### Statistical Analysis

Data expressed as mean±SEM of triplicate experiments. Statistical significance was calculated using one-way Analysis of Variance (ANOVA) with STATVIEW software. NIH ImageJ software was used for densitometry analysis. P<0.05 as significant compared to relative controls.

### Results

### Airspace Enlargement

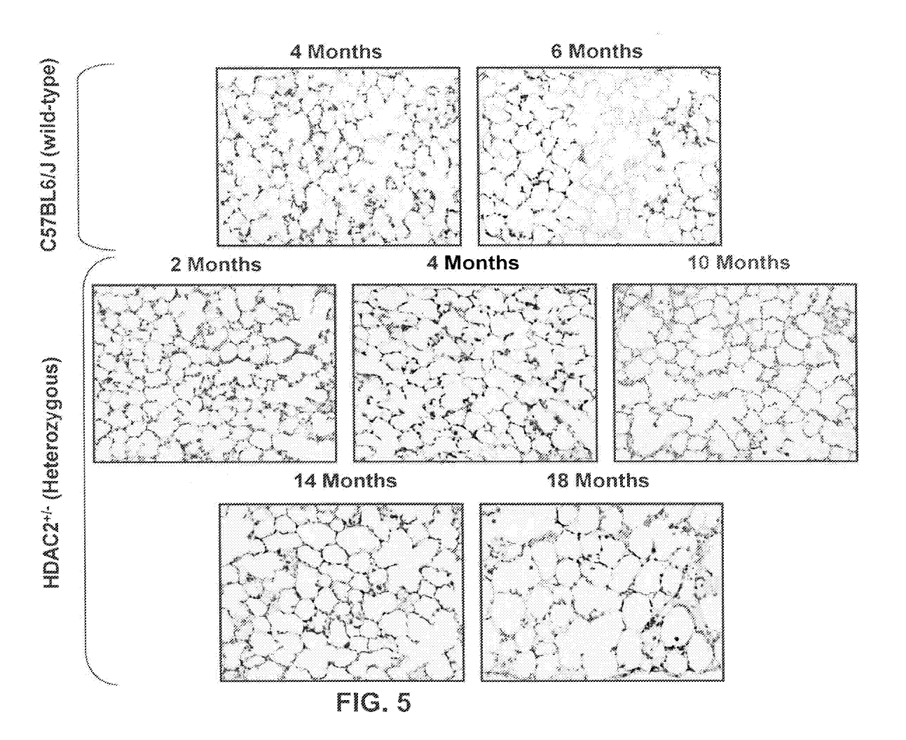

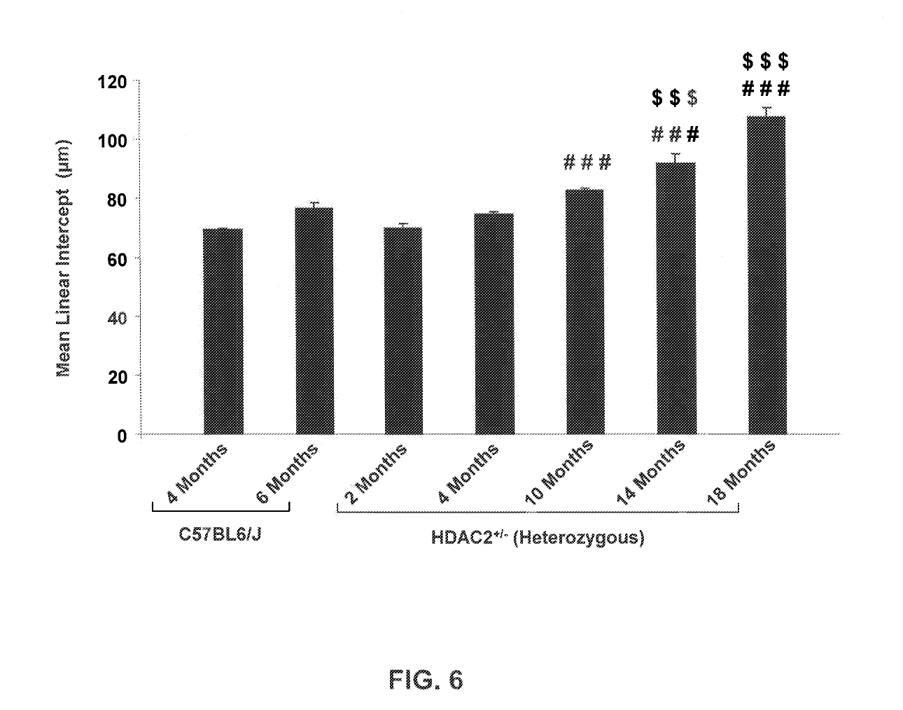

The mean linear intercept (airspace enlargement), which is one of the important characteristics of pulmonary emphysema/COPD, was measured in the lungs in HDAC2−/− mice. We compared the alveolar structure of naïve older 2-18 months old HDAC2−/− mice with that of wild-type controls (4-6 months). Alveolar airspace in HDAC2−/− mice was significantly enhanced age-dependently than in wild-type control mice (FIG. 5 and FIG. 6) suggesting spontaneous emphysema-like phenotype is associated with loss of HDAC2 gene expression.

### MMP9 Activity

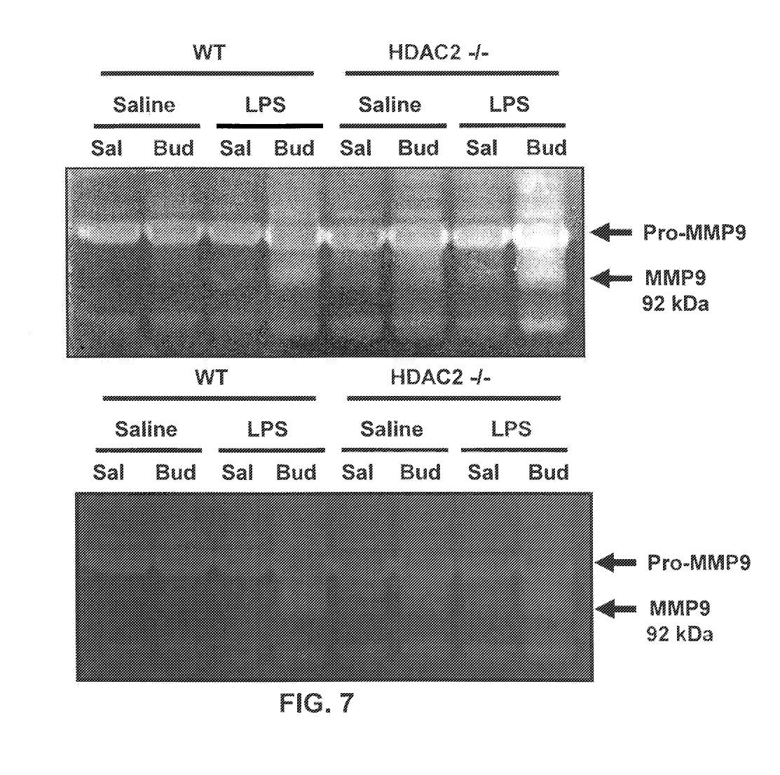

Matrix metalloproteinases (MMPs) particularly increased levels of MMP9 and MMP12 are involved in airspace enlargement/alveolar destruction (emphysema). Hence, it was determined whether the MMP9 activity was altered in HDAC2−/− mice with and without budesonide treatment. MMP9 activity was significantly higher in all HDAC2−/− groups compared to WT lung samples with only a minimal effect of budesonide treatment (FIG. 7). When the alveolar structure from 2-month to 18-month old mutant mice was compared to wild-type counterparts, alveolar spaces were greatly enlarged and the mutant mice showed increased MMP9 activity at 18-month of age compared to wild-type counterpart (data not shown), indicating loss of HDAC2 is a possible trigger for spontaneous emphysema-like phenotypes in mice. It is also possible that loss of HDAC2 is associated with increased cellular senescence by enhancing p21 and p53-dependent transactivation/transrepression of various genes leading to cellular senescence and apoptosis of lung cells (Yao et al., Am J Respir Cell Mol Biol 39: 7-18 (2008)). Hence, these data suggest that HDAC2 deficiency leads to steroid resistance in terms of attenuating lung inflammatory response.

### HDAC2−/− Mice are Less Responsive to Budesonide in Inhibiting LPS-Induced Lung Inflammation

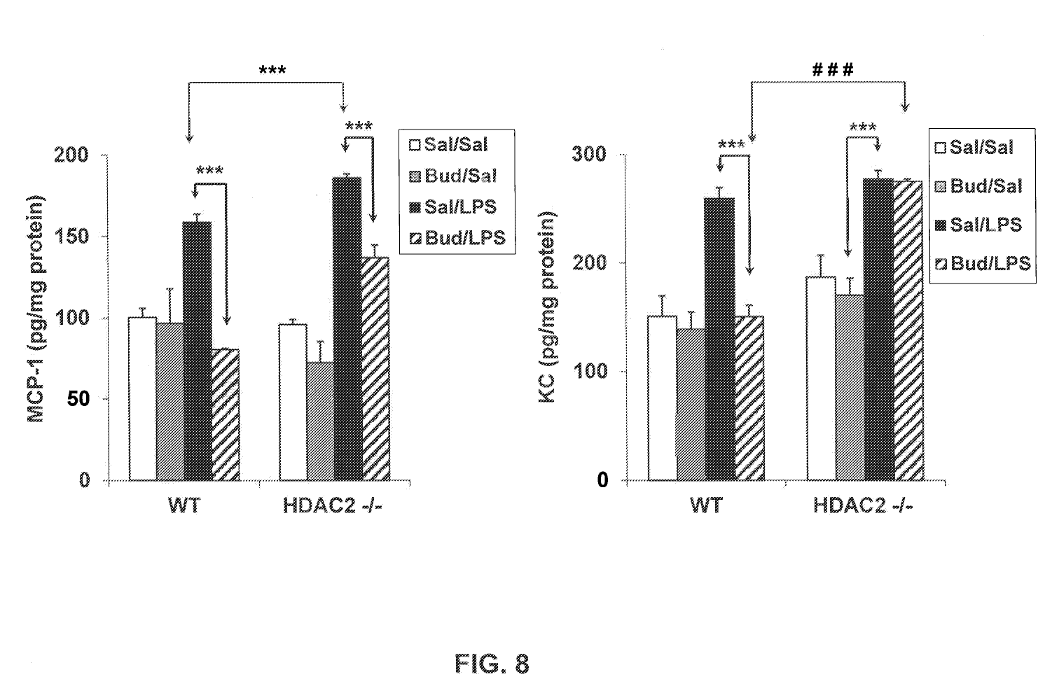

To determine whether HDAC2−/− would respond to steroid treatment in response to LPS-induced inflammation, age-matched WT and HDAC2−/− mice were treated with budesonide (3 mg/kg) via an intranasal route of administration for 3 days followed by exposure to aerosolized LPS for 6 minutes at 1 h post-last budesonide treatment. LPS was used here as a different lung inflammation-triggering agent rather than CS exposure since HDAC2 is inactivated and degraded in response to CS both in lungs in vivo, epithelial cells and macrophages in vitro (Adenuga et al., Am J Respir Cell Mol Biol 40: 464-473 (2009); Marwick et al., Am J Respir Cell Mol Biol, 31: 633-642 (2004); Yang et al., Am J Physiol Lung Cell Mol Physiol, 291: L46-57 (2006)). As expected, LPS aerosolization in WT mice had no effect on HDAC2 protein levels (data not shown). While budesonide pre-treatment completely blocked MCP-1 and KC release in WT mice, only about 30% reduction in MCP-1 levels was observed in HDAC2−/− mice. Budesonide however had no observable effect on KC release in response to LPS exposure in HDAC2−/− mice (FIG. 8).

### Nrf2−/− Mice are Not Responsive to Repress LPS-Induced Lung Inflammation by Budesonide

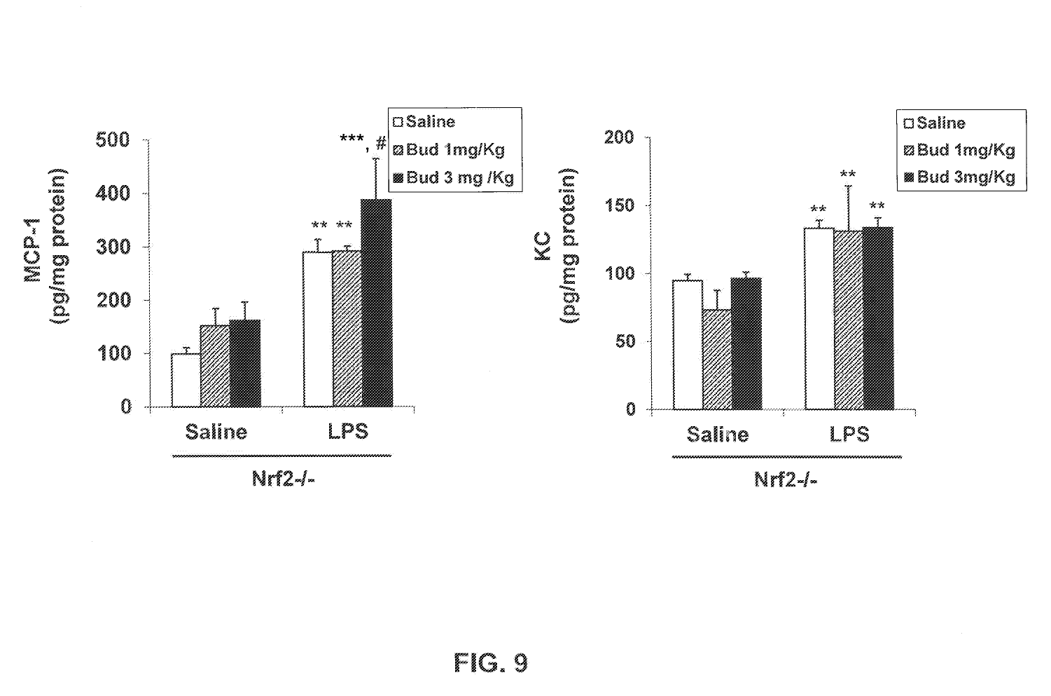

Decreased HDAC2 protein levels and deacetylase activity in naïve Nrf2−/− suggested that these mice may also exhibit resistance to steroid-mediated attenuation of lung inflammation. The release of MCP-1, a strong chemoattractant for monocytes and the neutrophil chemoattractant, KC, was measured in lung tissue. KC release was significantly increased in LPS-exposed Nrf2−/− with MCP-1 release significantly increased by 2.5-3.0 fold in LPS-exposed mice compared to saline-treated mice with no inhibitory effect with budesonide treatment (FIG. 9).

What is claimed is:

1. A method for the treatment of an inflammatory disorder in a subject comprising concomitantly administering to the subject a pharmaceutical formulation comprising:
an effective amount of a corticosteroid; and
an effective amount of a Nrf2 activator.

2. The method of claim 1, wherein the inflammatory disorder is chronic obstructive pulmonary disorder (COPD).

3. The method of claim 1, wherein the inflammatory disorder is selected from the group consisting of: acne vulgaris, asthma, autoimmune diseases, celiac disease, chronic prostatitis, glomerulonephritis, hypersensitivities, inflammatory bowel diseases, pelvic inflammatory disease, reperfusion injury, rheumatoid arthritis, sarcoidosis, transplant rejection, vasculitis, and interstitial cystitis.

4. The method of claim 1, wherein the Nrf2 activator is sulphoraphane.

5. The method of claim 1, wherein the Nrf2 activator is selected from the group consisting of: tert-butylhydroquinone (tBHQ), Oltipraz (4-methyl-5-(2-pyrazinyl)-3-dithiolethione), bardoxolone methyl, dihydro-CDDO-trifluoroethyl amide (dh404), resveratrol, chalcone, a chalcone derivative, anethole dithiolethione, 6-methylsulphinylhexyl isothiocyanate, curcumin, caffeic acid phenethyl ester, and 4′-bromoflavone.

6. The method of claim 1, wherein the corticosteroid is budesonide. The method of claim 1, wherein the corticosteroid is selected from the group consisting of: hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, aclometasone dipropionate, betamethasone valerate, betamethasone dipropionate, prednicarbate, clobetasone-17-butyrate, clobetasol-17-propionate fluocortolone caproate, fluocortolone pivalate, fluprednidene acetate, hydrocortisone-17-butyrate, 17-aceponate, 17-buteprate, and prednicarbate.

8. The method of claim 1, wherein the pharmaceutical formulation further comprises a PDE4 inhibitor.

9. The method of claim 8, wherein the PDE4 inhibitor is selected from the group consisting of: mesembrine, rolipram, ibudilast, piclamilast, luteolin, roflumilast, cilomilast, and diazepam.

10. The method of claim 1, wherein the pharmaceutical formulation further comprises an antioxidant.

11. The method of claim 10, wherein the antioxidant is selected from the group consisting of: N-acetylcysteine, N-acystelyn, thio-N-acetylcysteine, N-isobutyrylcysteine, glutathione, glutathione esters, S-carboxymthylcysteine, erdostein, fudosteine, thioredoxin, procysteine, ergothioneine, ascorbic acid, lipoic acid, uric acid, beta-carotene, retinol, melatonin, alpha-tocopherol and ubiquinol.

12. The method of claim 1, wherein the pharmaceutical formulation is an inhalable pharmaceutical formulation.

13. The method of claim 1, wherein the pharmaceutical formulation is a solid oral dosage form.

14. The method of claim 1, wherein the pharmaceutical formulation is an injectable pharmaceutical formulation.

15. A method for the treatment of a corticosteroid resistant inflammatory disorder in a subject comprising concomitantly administering to the subject:
a pharmaceutical formulation comprising an amount of corticosteroid to which the inflammatory disorder is resistant; and
a pharmaceutical formulation comprising an effective amount of a Nrf2 activator;
wherein the Nrf2 activator causes the amount of corticosteroid to be effective in treating the inflammatory disorder.

16. The method of claim 15, wherein the inflammatory disorder is chronic obstructive pulmonary disorder (COPD).

17. The method of claim 15, wherein the inflammatory disorder is selected from the group consisting of: acne vulgaris, asthma, autoimmune diseases, celiac disease, chronic prostatitis, glomerulonephritis, hypersensitivities, inflammatory bowel diseases, pelvic inflammatory disease, reperfusion injury, rheumatoid arthritis, sarcoidosis, transplant rejection, vasculitis, and interstitial cystitis.

18. The method of claim 15, wherein the Nrf2 activator is sulphoraphane.

19. The method of claim 15, wherein the Nrf2 activator is selected from the group consisting of: tert-butylhydroquinone (tBHQ), Oltipraz (4-methyl-5-(2-pyrazinyl)-3-dithiolethione), bardoxolone methyl, dihydro-CDDO-trifluoroethyl amide (dh404), resveratrol, chalcone, a chalcone derivative, anethole dithiolethione, 6-methylsulphinylhexyl isothiocyanate, curcumin, caffeic acid phenethyl ester, and 4′-bromoflavone.

20. The method of claim 15, wherein the corticosteroid is budesonide.

21. The method of claim 15, wherein the corticosteroid is selected from the group consisting of: hydrocortisone, hydrocortisone acetate, cortisone acetate, tixocortol pivalate, prednisolone, methylprednisolone, prednisone, triamcinolone acetonide, triamcinolone alcohol, mometasone, amcinonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, hydrocortisone-17-butyrate, 17-aceponate, 17-buteprate, and prednicarbate.

22. The method of claim 15, furthering comprising concomitant administration of a PDE4 inhibitor,

23. The method of claim 22, wherein the pharmaceutical formulation comprising the Nrf2 activator comprises the PDE4 inhibitor.

24. The method of claim 22, wherein the PDE4 inhibitor is selected from the group consisting of: mesembrine, rolipram, ibudilast, piclamilast, luteolin, roflumilast, cilomilast, and diazepam.

25. The method of claim 15, furthering comprising concomitant administration of an antioxidant.

26. The method of claim 25, wherein the pharmaceutical formulation comprising the Nrf2 activator comprises the antioxidant.

27. The method of claim 25, wherein the antioxidant is selected from the group consisting of: N-acetylcysteine, nacystelyn, thio-N-acetylcysteine, glutathione, ascorbic acid, lipoic acid, uric acid, beta-carotene, retinol, melatonin, alpha-tocopherol and ubiquinol.

28. The method of claim 15, wherein the pharmaceutical formulation comprising the Nrf2 activator is an inhalable pharmaceutical formulation.

29. The method of claim 15, wherein the pharmaceutical formulation comprising the Nrf2 activator is a solid oral dosage form.

30. The method of claim 15, wherein the pharmaceutical formulation comprising the Nrf2 activator is an injectable pharmaceutical formulation.

31. The method of claim 15, wherein the pharmaceutical formulation comprising the corticosteroid is an inhalable pharmaceutical formulation.

32. The method of claim 15, wherein the pharmaceutical formulation comprising the corticosteroid is a solid oral dosage form.

33. The method of claim 15, wherein the pharmaceutical formulation comprising the corticosteroid is an injectable pharmaceutical formulation.

34. A pharmaceutical formulation comprising:
an effective amount of corticosteroid; and
an effective amount of a Nrf2 activator.

35. The pharmaceutical formulation of claim 34, wherein the Nrf2 activator is sulphoraphane.

36. The pharmaceutical formulation of claim 34, wherein the Nrf2 activator is selected from the group consisting of: tert-butylhydroquinone (tBHQ), sulforaphane, Oltipraz (4-methyl-5-(2-pyrazinyl)-3-dithiolethione), bardoxolone methyl, dihydro-CDDO-trifluoroethyl amide (dh404), resveratrol, chalcone, a chalcone derivative, anethole dithiolethione, 6-methylsulphinylhexyl isothiocyanate, curcumin, caffeic acid phenethyl ester, and 4′-bromoflavone.

37. The pharmaceutical formulation of claim 34, wherein the corticosteroid is budesonide.

38. The pharmaceutical formulation of claim 34, wherein the corticosteroid is selected from the group consisting of: hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, hydrocortisone-17-butyrate, 17-aceponate, 17-buteprate, and prednicarbate.

39. The pharmaceutical formulation of claim 34, wherein the pharmaceutical formulation further comprises a PDE4 inhibitor.

40. The pharmaceutical formulation of claim 39, wherein the PDE4 inhibitor is selected from the group consisting of: mesembrine, rolipram, ibudilast, piclamilast, luteolin, roflumilast, cilomilast, and diazepam.

41. The pharmaceutical formulation of claim 34, wherein the pharmaceutical formulation further comprises an antioxidant.

42. The pharmaceutical formulation of claim 41, wherein the antioxidant is selected from the group consisting of: N-acetylcysteine, N-acystelyn, thio-N-acetylcysteine, N-isobutyrylcysteine, glutathione, glutathione esters, S-carboxymthylcysteine, erdostein, fudosteine, thioredoxin, procysteine, ergothioneine, ascorbic acid, lipoic acid, uric acid, beta-carotene, retinol, melatonin, alpha-tocopherol and ubiquinol.

43. The pharmaceutical formulation of claim 34, wherein the pharmaceutical formulation is an inhalable pharmaceutical formulation.

44. The pharmaceutical formulation of claim 34, wherein the pharmaceutical formulation is a solid oral dosage form.

45. The pharmaceutical formulation of claim 34, wherein the pharmaceutical formulation is an injectable pharmaceutical formulation.

46. A method for increasing cellular levels of HDAC2 in a cell comprising,
providing the cell, and
contacting the cell with an Nrf2 activator;
wherein the Nrf2 activator causes an increase in the cellular levels of HDAC2.

47. The method of claim 46, wherein the method is performed in vivo.

48. The method of claim 46, wherein the method is performed in vitro.

49. The method of claim 46, wherein the Nrf2 activator is selected from the group consisting of: sulphoraphane, tert-butylhydroquinone (tBHQ), sulforaphane, Oltipraz (4-methyl-5-(2-pyrazinyl)-3-dithiolethione), bardoxolone methyl, dihydro-CDDO-trifluoroethyl amide (dh404), resveratrol, chalcone, a chalcone derivative, anethole dithiolethione, 6-methylsulphinylhexyl isothiocyanate, curcumin, caffeic acid phenethyl ester, and 4′-bromoflavone.

50. A method for increasing cellular levels of HDAC2 in a cell comprising,
providing the cell, and
contacting the cell with a nucleic acid that causes expression of an Nrf2 polypeptide in the cell;
wherein the expression of the Nrf2 polypeptide causes an increase in the cellular levels of HDAC2.

51. The method of claim 50, wherein the method is performed in vivo.

52. The method of claim 50, wherein the method is performed in vitro.

53. The method of claim 50, wherein the nucleic acid is a vector.

54. The method of claim 50, wherein the nucleic acid comprises a nucleic acid sequence encoding a polypeptide which is at least 80% identical to SEQ ID NO:2.

55. The method of claim 50, wherein the nucleic acid comprises a nucleic acid sequence encoding a polypeptide which is at least 90% identical to SEQ ID NO:2.

56. The method of claim 50, wherein the nucleic acid comprises a nucleic acid sequence encoding a polypeptide which is at least 95% identical to SEQ ID NO:2.

57. A method for the treatment or prevention of a disease related to reduced cellular levels of histone deacetylase 2 (HDAC2) in a subject, comprising, providing to the subject a molecular compound which acts as an activator of Nuclear factor (erythroid-derived 2)-like 2 (Nrf2).

58. The method of claim 57, wherein the molecular compound is provided in a pharmaceutical formulation.

59. The method of claim 58, wherein the pharmaceutical formulation is an inhaled pharmaceutical formulation.

60. The method of claim 57, wherein the molecular compound is sulforaphane.

61. The method of claim 58, wherein the molecular compound is selected from tert-butylhydroquinone (tBHQ), sulforaphane, Oltipraz (4-methyl-5-(2-pyrazinyl)-3-dithiolethione), bardoxolone methyl, dihydro-CDDO-trifluoroethyl amide (dh404), resveratrol, chalcone, a chalcone derivative, anethole dithiolethione, 6-methylsulphinylhexyl isothiocyanate, curcumin, caffeic acid phenethyl ester, and 4′-bromoflavone.

62. The method of claim 57, further comprising providing to the subject a corticosteroid.

63. The method of claim 62, wherein the corticosteroid is selected from the group consisting of: hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, hydrocortisone-17-butyrate, 17-aceponate, 17-buteprate, and prednicarbate.

64. The method of claim 57, wherein the disease related to reduced cellular levels of HDAC2 is chronic obstructive pulmonary disorder (COPD).

65. The method of claim 64, wherein the COPD is resistant to treatment with corticosteroids.

66. The method of claim 57, wherein the disease related to reduced cellular levels of HDAC2 is asthma.

67. The method of claim 66, wherein the asthma is resistant to treatment with corticosteroids.

68. The method of claim 57, wherein the disease related to reduced cellular levels of HDAC2 is rheumatoid arthritis.

69. The method of claim 68, wherein the rheumatoid arthritis is resistant to treatment with corticosteroids.

70. The method of claim 57, wherein the disease related to reduced cellular levels of HDAC 2 is inflammatory bowel disease.

71. The method of claim 70, wherein the inflammatory bowel disease is resistant to treatment with corticosteroids.

72. A method for the treatment or prevention of COPD or asthma in a subject, comprising, administering to the subject a nucleic acid sequence which causes expression of Nrf2 in the lung cells of the subject.

73. The method of claim 72, wherein the nucleic acid sequence is a nucleic acid sequence encoding a protein having an amino acid sequence which is 80% identical to SEQ ID NO: 2.
